(19) United States
(12) Patent Application Publication
(10) Pub. No.: US 2024/0218033 A1
(43) Pub. Date: Jul. 4, 2024

(54) PREVENTION AND TREATMENT OF CHEMOTHERAPY-INDUCED NEUROPATHIC PAIN

(71) Applicant: HOBA THERAPEUTICS APS, København N (DK)

(72) Inventors: Kenneth PETERSEN, Frederiksberg (DK); Gordon MUNRO, Skovlunde (DK); Torsten Meldgaard MADSEN, København Ø (DK)

(21) Appl. No.: 18/558,570

(22) PCT Filed: May 5, 2022
(86) PCT No.: PCT/EP2022/062083

§ 371 Date: Nov. 2, 2023

(30) Foreign Application Priority Data
May 6, 2021 (EP) 21172472.9

(51) Int. Cl. C07K 14/47; A61K 31/337; A61K 38/17; A61K 48/00; A61P 25/02

(52) U.S. Cl. CPC C07K 14/4705; A61K 31/337; A61K 38/1709; A61K 48/0033; A61P 25/02

(57) ABSTRACT

The present disclosure relates to Meteorin and its use in prevention and/or treatment of chemotherapy-induced neuropathic pain. Neuropathic pain arising as a result of treatment with a chemotherapeutic may be treated by administration of Meteorin to the patient. Meteorin may also be used in prophylactic treatment to prevent neuropathic pain from developing as a result of treatment with a chemotherapeutic.

### TECHNICAL FIELD

The present invention relates to Meteorin and its use in prevention and/or treatment of chemotherapy-induced neuropathic pain.

### BACKGROUND

Cancer is one of the main causes of death across the world and despite the huge efforts to implement novel chemotherapy strategies, these diseases remain a major health concern, with millions of new cases reported each year.

Treatment with chemotherapy leads to improved cancer survival, but is also often responsible for serious side effects, which reduces the quality of life for cancer patients considerably.

Important anti-cancer agents, including platinum-based agents, taxanes and vinca alkaloids, are known to cause neurotoxicity to the peripheral nervous system, thereby causing neuropathic pain, with symptoms such as allodynia, hyperalgesia and spontaneous pain. Chemotherapy-induced neuropathic pain (CINP), is one of the most severe side effects of chemotherapy. CINP causes long-term discomfort to the patients with the side effects potentially lasting many years after discontinuation of treatment, which reduce the quality of life of the cancer survivors. Chemotherapy-induced neuropathy and pain are thus the most frequent non-hematological dose-limiting side effects of anti-cancer drugs. If the dose is too high, side effects will be intolerable to the person receiving it, whereas low doses will result in ineffective treatment of the underlying cancer/disease. Consequently, the efficacy of many anticancer drugs is suboptimal at doses where side effects are acceptable for most patients. Thus, CINP symptoms of chemotherapeutic treatment potentially leads to reduction of the chemotherapeutic dosage or discontinuation of treatment, which consequently leads to decreased survival rates.

Safe and effective therapies to prevent or treat chemotherapy-induced neuropathic pain are still an unmet clinical need with no approved therapies. Drugs normally used against chronic pain conditions, such as gabapentin, tricyclic antidepressants, and opioids, are poorly effective and associated with numerous side effects.

Hence, there is a high need of preventive and therapeutic strategies for treatment of chemotherapy-induced neuropathic pain, preferably with none or only minor side effects that do not affect the general health of the patients.

Meteorin is an endogenous protein which has previously been demonstrated to be a survival factor for neurons (WO 2005/095450). WO 2012/041328 describes the use of Meteorin for treatment of allodynia, hyperalgesia, spontaneous pain, and phantom pain based on findings in animal models of nerve injury.

### SUMMARY

The inventors of the present disclosure have surprisingly found that administration of Meteorin prior to, simultaneously or intermittently with chemotherapy treatment results in prevention of chemotherapy-induced neuropathic pain (CINP). Thus, by administering Meteorin in conjunction with chemotherapy, CINP can be prevented. CINP is a severe side effect of chemotherapy, as the symptoms in addition to causing long-term discomfort to the patients, also potentially mandate a reduction in the dosage amount of the chemotherapeutics or to discontinuation of treatment, which consequently leads to decreased survival rates. Hence, the present invention provides means for improving cancer therapy by allowing the use of a higher dosage of the chemotherapeutics with reduced risk of development of neuropathic pain.

In one aspect, the present invention relates to an isolated polypeptide for use in treatment or prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In a second aspect, the present invention relates to an isolated nucleic acid molecule for use in treatment or prevention of chemotherapy-induced neuropathic pain in a subject, said nucleic acid molecule comprising a nucleic acid sequence coding for a polypeptide comprising an amino acid sequence selected from the group consisting of:
a. the amino acid sequence of SEQ ID NO: 3; a.
b. a biologically active sequence variant of the amino acid sequence of SEQ ID NO: 3, wherein the variant has at least 70% sequence identity to SEQ ID NO: 3.

In a further aspect, the present invention relates to a vector for use in treatment or prevention of chemotherapy-induced neuropathic pain in a subject, said vector comprising a polynucleotide coding for a polypeptide according to any of the claims 1 to 6.

In a further aspect, the present invention relates to a method for reducing glutamine synthetase expression in dorsal root ganglion in a subject in need thereof the method comprising administering a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3 thereby reducing expression of glutamine synthetase in dorsal root ganglion.

In a further aspect, the present invention relates to a method for reducing Connexin 43 expression in dorsal root ganglion in a subject in need thereof the method comprising administering a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3 thereby reducing expression of Connexin 43 in dorsal root ganglion.

### DESCRIPTION OF DRAWINGS

FIG. 1: Study design preventive paradigm. rmMeteorin 0.5 mg/kg or 1.8 mg/kg was administered s.c. on days 1, 3, 5, 7, and 9 (D1, D3, D5, D7, and D9). Paclitaxel was administered i.p. on days 2, 4, 6, and 8 (D2, D4, D6, and D8). Paclitaxel (PTX); intraperitoneal (i.p.); subcutaneous (s.c.); spinal cord (SC); dorsal root ganglia (DRG).

FIG. 2: Meteorin prevents paclitaxel-induced mechanical hypersensitivity in adult C57BI6J mice. Hindpaw paw withdrawal thresholds (PWTs) were measured at baseline (BL) and then routinely throughout the experimental duration until Day 57 using von Frey filaments. Paclitaxel treatment was preceded by a s.c. injection of 0.5 mg/kg (grey squares) or 1.8 mg/kg (black triangles) or rmMeteorin (MTRN) or vehicle (n=8 group) (white circles), and then a subsequent 4 additional injections of each treatment as indicated by arrows. The development of mechanical hypersensitivity was essentially prevented by rmMeteorin (0.5 mg/kg and 1.8 mg/kg) compared with vehicle treatment. *p<0.05; **p<0.01; ***p<0.001, ****p<0.0001 vs vehicle, ANOVA mixed-effects model followed by Tukey post hoc test. Data are shown as mean±SEM.

FIG. 3: Meteorin prevents paclitaxel-induced increases in satellite glial cell density and gap junction formation.

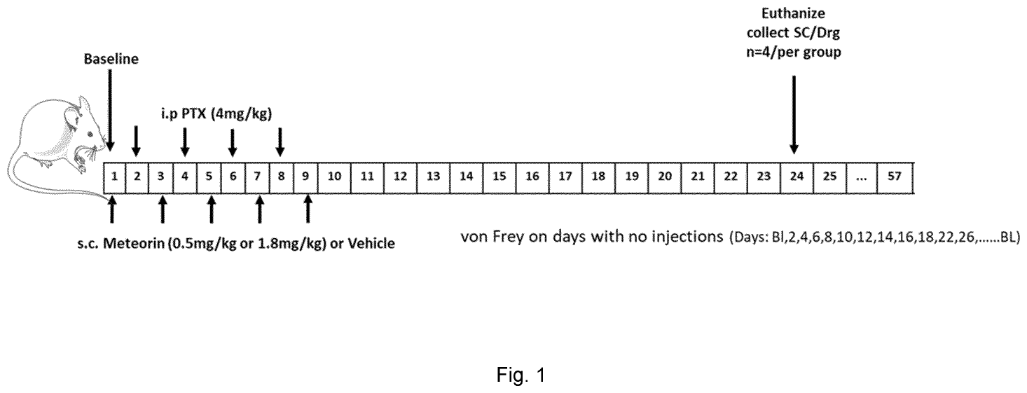

Female mice were administered paclitaxel (4 mg/kg, i.p.) every other day for 4 days on days 2,4,6,8 alternated by injection of rmMeteorin (0.5 mg/kg or 1.8 mg/kg, s.c.) or vehicle on days 1,3,5,7,9 (as shown in FIG. 1). At day 24 mice were killed and dorsal root ganglion (DRG) tissue removed for immunohistochemical processing with antibodies to peripherin (which was used to identify neuronal cell bodies—not shown), glutamine synthetase (GS) and Connexin 43 (Con43). Mean grey intensity (MGI) was expressed as a function of specific staining for each respective antibody per μm². *p<0.05, **p<0.01 vs Vehicle one-way ANOVA with Tukey multiple comparisons. Data are shown as mean±SEM.

FIG. 4: Pre-emptive Meteorin treatment prevents paclitaxel-induced loss of hindpaw intraepidermal nerve fibres. Female mice were administered paclitaxel (4 mg/kg, i.p.) every other day for 4 days on days 2, 4, 6, and 8 (D2, D4, D6, and D8) alternated by injection of rmMeteorin (0.5 mg/kg or 1.8 mg/kg, s.c.) or Vehicle on days 1, 3, 5, 7, and 9 (D1, D3, D5, D7, and D9). PGP9.5 expression was used as a specific marker to calculate intraepidermal nerve fibres (IENFs) density calculated from the number of IENFs found crossing the basement membrane (arrows) and normalized to the width of the epidermis (mm). *p<0.05, **p<0.01, ***p<0.001, ****p<0.0001 one-way ANOVA vs vehicle with Tukey multiple comparisons. Data are shown as mean±SEM.

FIG. 5: Study design treatment paradigm. Paclitaxel was administered i.p. to separate cohorts of male and female mice on days 2, 4, 6, and 8 (D2, D4, D6, and D8). rmMeteorin 0.5 mg/kg or 1.8 mg/kg was administered s.c. on days 10, 12, 14, 16, and 18 (D10, D12, D14, D16, and D18). Paclitaxel (PTX); intraperitoneal (i.p.); subcutaneous (s.c.); spinal cord (SC); dorsal root ganglia (DRG).

FIG. 6: Meteorin reverses paclitaxel-induced mechanical hypersensitivity in adult C57BI6J mice.

According to the study plan of FIG. 5, separate cohorts of male and female mice were administered an i.p. injection of 4 mg/kg paclitaxel (PTX) every other day for a cumulative dosage of 16 mg/kg (grey box). PWTs were measured at baseline (BL) and then routinely throughout the experimental duration until Day 54 using von Frey filaments. Paclitaxel treatment was followed by 5 repeated s.c. injections of 0.5 mg/kg (grey squares) or 1.8 mg/kg (black triangles) of rmMeteorin (MTRN) or vehicle (n=8 group for each sex) at the times indicated by arrows.

Data was combined for male and female treatment groups. Mechanical hypersensitivity was reduced and resolved more quickly by rmMeteorin treatment (0.5 mg/kg and 1.8 mg/kg) compared with vehicle treatment. *p<0.05; **p<0.01; ***p<0.001, ****p <0.0001 vs vehicle, ANOVA mixed-effects model followed by Tukey post hoc test. Data are shown as mean±SEM.

FIG. 7: Meteorin reverses paclitaxel-induced increases in satellite glial cell density and gap junction formation.

Female mice (upper panels) or male mice (lower panels) were administered paclitaxel (4 mg/kg, i.p.) every other day for 4 days on days 2, 4, 6, 8. Subsequently rmMeteorin (0.5 mg/kg or 1.8 mg/kg, s.c.) or vehicle was administered on days 10, 12, 14, 16, 18 (as shown in FIG. 5). At day 24 mice were killed and dorsal root ganglion (DRG) tissue removed for immunohistochemical processing with antibodies to peripherin (which was used to identify neuronal cell bodies—not shown), glutamine synthetase (GS) and Connexin43 (Con43). Mean grey intensity (MGI) was expressed as a function of specific staining for each respective antibody per μm². *p<0.05, **p<0.01 vs Vehicle one-way ANOVA with Tukey multiple comparisons. Data are shown as mean+SEM.

FIG. 8: CLUSTAL W (1.82) multiple sequence alignment of Meteorin.

A) Alignment of Meteorin precursors from human (SEQ ID NO: 2), rat (SEQ ID NO: 8), and mouse (SEQ ID NO: 5). B) Alignment of mature Meteorin from human (SEQ ID NO: 3), rat (SEQ ID NO: 9), and mouse (SEQ ID NO: 6). C) Mature Meteorin, consensus sequence (SEQ ID NO: 11) generated from fully conserved residues in the human, mouse and rat sequences. X represents any of the 21 naturally occurring amino acid encoded by DNA.

### DETAILED DESCRIPTION

### Definitions

As used herein “a biocompatible capsule” means that the capsule, upon implantation in a host mammal, does not elicit a detrimental host response sufficient to result in the rejection of the capsule or to render it inoperable, for example through degradation.

As used herein, a “coding sequence” is a polynucleotide sequence which is transcribed and translated into a polypeptide.

As used herein, the term “expression vectors” refers to vectors that are capable of directing the expression of genes to which they are operably-linked. In general, expression vectors of utility in recombinant DNA techniques are often in the form of plasmids.

As used herein “an immunoisolatory capsule” means that the capsule upon implantation into a mammalian host minimizes the deleterious effects of the host's immune system on the cells within its core.

By a “mammalian promoter” is intended a promoter capable of functioning in a mammalian cell.

“Meteorin”, as used herein, refers to polypeptides having the amino acid sequences of substantially purified Meteorin obtained from any species, particularly mammalian, including chimpanzee, bovine, ovine, porcine, murine, equine, and preferably human, from any source whether natural, synthetic, semi-synthetic, or recombinant. The term also refers to biologically active fragments of Meteorin obtained from any of these species, as well as to biologically active sequence variants of these and to proteins subject to posttranslational modifications.

As used herein, the term “operatively-linked” is intended to mean that the nucleotide sequence of interest is linked to the regulatory sequence(s) within a recombinant expression vector, in a manner that allows for expression of the nucleotide sequence (e.g., in an in vitro transcription/translation system or in a host cell when the vector is introduced into the host cell).

As used herein, the term “regulatory sequence” is intended to include promoters, enhancers and other expression control elements (e.g., polyadenylation signals).

“Sequence identity”: A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. The ClustalW software is available as a ClustalW WWW Service at the European Bioinformatics Institute from http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similarly be used to align nucleotide sequences. Sequence identities may be calculated in a similar way as indicated for amino acid sequences.

The term “subject” used herein is taken to mean any mammal to which Meteorin polypeptide or polynucleotide, therapeutic cells or biocompatible capsules may be administered. Subjects specifically intended for treatment with the method of the invention include humans, as well as nonhuman primates, sheep, horses, cattle, goats, pigs, dogs, cats, rabbits, guinea pigs, hamsters, gerbils, rats and mice, as well as the organs, tumors, and cells derived or originating from these hosts.

“Treatment” can be performed in different ways, including curative and/or ameliorating. Curative treatment generally aims at curing a clinical condition, which is already present in the treated individual. Ameliorating treatment generally means treating in order to improve, in an individual, an existing clinical condition.

The term “prevention” as used herein refers to preventing a clinical condition or reducing the risk of contracting the condition or reducing the extent of the condition. Prevention may also be referred to herein as prophylactic treatment or pre-emptive treatment.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

Chemotherapy and Neuropathic pain

Chemotherapy is a type of cancer treatment, that uses one or more anticancer drugs to treat cancer systemically. Treatment with chemotherapy leads to prolonged life but is also responsible for serious side effects. Antineoplastic agents in chemotherapy are designed to eliminate rapidly dividing cancer cells, but they can also damage healthy structures, including the peripheral nervous system.

One of the more severe side effects of chemotherapy is chemotherapy-induced neuropathic pain, which is a category of pain that includes several forms of pain deriving from dysfunction of the peripheral nervous system and/or the central system caused by neurotoxicity of the chemotherapy. Symptoms of neuropathic pains include sensations of burning, tingling, electricity, pins and needles, paresthesia, dysesthesia, stiffness, numbness in the extremities, feelings of bodily distortion, allodynia (pain evoked by stimulation that is normally innocuous), hyperalgesia (abnormal sensitivity to pain), hyperpathia (an exaggerated pain response persisting long after the pain stimuli cease), and spontaneous pain.

CINP afflicts between 30% and 40% of patients undergoing chemotherapy. The prevalence of these symptoms is highest in the first month after the completion of chemotherapy at 68.1%, but as many as 30% of patients still report CINP symptoms six months after the completion of chemotherapy. The severity of the symptoms is generally proportional to the dose of the treatment drug received, and the severity of the symptoms may warrant a reduction in the chemotherapy dosage.

CINP may arise from chemotherapeutic treatment of various cancer indications including but not limited to ovarian cancer, breast cancer, cancers of the GI tract such as esophageal cancer, pancreatic cancer, leukemia, Hodgkin's disease, Wilms' tumor, neuroblastoma, testicular cancer, bladder cancer, lung cancer and multiple myeloma. Thus, in one embodiment, chemotherapy-induced neuropathic pain is induced by chemotherapeutic treatment of ovarian cancer, breast cancer, esophageal cancer, pancreatic cancer, leukemia, Hodgkin's disease, Wilms' tumor, neuroblastoma, testicular cancer, bladder cancer, lung cancer or multiple myeloma. In another embodiment, chemotherapy-induced neuropathic pain is induced by chemotherapeutic treatment of ovarian cancer, breast cancer, esophageal cancer, pancreatic cancer, leukemia, Hodgkin's disease, Wilms' tumor, neuroblastoma, testicular cancer, bladder cancer, or multiple myeloma.

CINP may be induced by different anticancer agents, including but not limited to platinum-based anticancer agents, such as carboplatin, cisplatin, and oxaliplatin; taxanes, such as paclitaxel and docetaxel; epothilones, such as ixabepilone; vinca alkaloids, such as vincristine, vinblastine, and vinorelbine; proteasome inhibitors, such as bortezomib; and immunomodulatory anticancer agents, such as thalidomide. Thus, in one embodiment, the chemotherapy-induced neuropathic pain is induced by treatment with an anticancer agent selected from the group consisting of platinum-based anticancer agents, taxanes, epothilones, vinca alkaloids, proteasome inhibitors, and immunomodulatory anticancer agents.

In one embodiment, the chemotherapy-induced neuropathic pain is induced by treatment with a platinum-based anticancer agent, such as induced by treatment with carboplatin, cisplatin and/or oxaliplatin. In one embodiment, the chemotherapy-induced neuropathic pain is induced by treatment with a taxane, such as induced by treatment with paclitaxel and/or docetaxel. In one embodiment, the chemotherapy-induced neuropathic pain is induced by treatment with an epothilone, such as induced by treatment with ixabepilone. In one embodiment, the chemotherapy-induced neuropathic pain is induced by treatment with a vinca alkaloid, such as induced by treatment with vincristine, vinblastine, and/or vinorelbine. In one embodiment, the chemotherapy-induced neuropathic pain is induced by treatment with a proteasome inhibitor, such as induced by treatment with bortezomib. In one embodiment, the chemotherapy-induced neuropathic pain is induced by treatment with an immunomodulatory anticancer agent, such as induced by treatment with thalidomide.

The anticancer agent may be given as single-agent treatment, or in a combinational treatment regime, wherein the anticancer agent is administered in combination with another anticancer agent.

Chemotherapy-induced neuropathic pain manifests initially as an acute pain syndrome, with sensory symptoms arising during or just after drug administration, and progress to a chronic neuropathy after repetitive chemotherapy treatment cycles. Regarding the duration of sensory symptoms, acute neuropathy generally subsides between treatments, while chronic neuropathy can persist for months or years, considerably reducing the quality of life of cancer survivors.

The symptoms resulting from CINP may vary and include burning, tingling, electricity, pins and needles, paresthesia, dysesthesia, stiffness, numbness in the extremities, feelings of bodily distortion, allodynia, hyperalgesia, hyperpathia, and/or spontaneous pain. Thus, in one embodiment, the chemotherapy-induced neuropathic pain results in burning, tingling, electricity, pins and needles, paresthesia, dysesthesia, stiffness, numbness in the extremities, feelings of bodily distortion, allodynia, hyperalgesia, hyperpathia, and/or spontaneous pain.

In one embodiment, the chemotherapy-induced neuropathic pain results in burning, tingling, electricity, pins and needles, paresthesia, dysesthesia, stiffness, numbness in the extremities, feelings of bodily distortion.

In one embodiment, the chemotherapy-induced neuropathic pain results in allodynia, hyperalgesia, hyperpathia, and/or spontaneous pain. In one embodiment, the chemotherapy-induced neuropathic pain results in allodynia. In one embodiment, the chemotherapy-induced neuropathic pain results in hyperalgesia. In one embodiment, the chemotherapy-induced neuropathic pain results in hyperpathia. In one embodiment, the chemotherapy-induced neuropathic pain results in spontaneous pain.

Treatment and/or prevention of chemotherapy-induced neuropathic pain

Safe and effective therapies to prevent or treat chemotherapy-induced neuropathic pain are still an unmet clinical need. Drugs normally effective against chronic pain conditions, such as gabapentin, tricyclic antidepressants and opioids, are poorly effective and associated with numerous side effects.

Hence, there is a high need of preventive and therapeutic strategies for treatment of chemotherapy-induced neuropathic pain, preferably with only minor side effects not affecting the general health of the patients. The present invention provides treatment and/or prevention of CINP by administration of Meteorin to the subject receiving chemotherapeutic treatment. Thus, in one embodiment the present invention relates to Meteorin for use in the treatment and/or prevention of chemotherapy-induced neuropathic pain. In one embodiment the present invention relates to Meteorin for use in the treatment of chemotherapy-induced neuropathic pain.

In one embodiment, the present disclosure provides an isolated polypeptide for use in treatment and/or prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one embodiment, the present invention relates to a method for treatment and/or prevention of chemotherapy-induced neuropathic pain, the method comprising administering a therapeutically effective amount of an isolated polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3,
   to a subject in need thereof

In one embodiment, the present disclosure provides use of an isolated polypeptide for the manufacture of a medicament for the treatment and/or prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

As demonstrated in examples 1, 2, and 3 of the present disclosure, administration of Meteorin prior to and/or intermittently with administration of the chemotherapeutic agent provides reversal of the chemotherapy-induced neuropathic pain. Thus, in a preferred embodiment the present invention relates to Meteorin for use in prevention of chemotherapy-induced neuropathic pain.

In one embodiment, the present disclosure provides an isolated polypeptide for use in prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one embodiment, the present invention relates to a method for prevention of chemotherapy-induced neuropathic pain, the method comprising administering a therapeutically effective amount of an isolated polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3,

to a subject in need thereof

In one embodiment, the present disclosure provides use of an isolated polypeptide for the manufacture of a medicament for the prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

In one embodiment the therapeutic effect of said treatment ameliorates at least one symptom of chemotherapy-induced neuropathic pain. The at least one symptom may be selected from the group consisting of burning, tingling, electricity, pins and needles, paresthesia, dysesthesia, stiffness, numbness in the extremities, feelings of bodily distortion, allodynia, hyperalgesia, hyperpathia, and/or spontaneous pain. In one embodiment the therapeutic effect of said treatment ameliorates at least one symptom selected from the group consisting of burning, tingling, electricity, pins and needles, paresthesia, dysesthesia, stiffness, numbness in the extremities, feelings of bodily distortion. In one embodiment the therapeutic effect of said treatment ameliorates at least one symptom selected from the group consisting allodynia, hyperalgesia, hyperpathia, and/or spontaneous pain.

In one embodiment the therapeutic effect of said treatment ameliorates allodynia. In one embodiment, the allodynia is thermal allodynia, cold allodynia, heat allodynia and/or mechanical allodynia.

In one embodiment the therapeutic effect of said treatment ameliorates hyperalgesia. In one embodiment, the hyperalgesia is mechanical hyperalgesia.

In one embodiment the therapeutic effect of said treatment ameliorates hyperpathia.

In one embodiment the therapeutic effect of said treatment ameliorates spontaneous pain.

### Administration and Formulation

Meteorin polypeptides may be administered in any manner, which is medically acceptable. This may include injections, by parenteral routes such as intravenous, intravascular, intraarterial, subcutaneous, intramuscular, intratumor, intraperitoneal, intraventricular, intraepidural, intrathecal, intracerebroventricular, intracerebral, or others as well as nasal, or topical. Slow-release administration is also specifically included in the invention, by such means as depot injections or erodible implants.

Administration of Meteorin according to this invention may be achieved using any suitable delivery means, including: injection, either subcutaneously, intravenously, intra-arterially, intramuscularly, intrathecally or to other suitable site; pump (see, e.g., Annals of Pharmacotherapy, 27:912 (1993); Cancer, 41:1270 (1993); Cancer Research, 44:1698 (1984), incorporated herein by reference); microencapsulation (see, e.g., U.S. Pat. Nos. 4,352,883; 4,353,888; and 5,084,350, herein incorporated by reference), slow-release polymer implants (see, e.g., Sabel, U.S. Pat. No. 4,883,666, incorporated herein by reference); encapsulated cells (see, “Biocompatible capsules”); unencapsulated cell grafts (see, e.g., U.S. Pat. Nos. 5,082,670 and 5,618,531, each incorporated herein by reference); and inhalation.

Administration may be by periodic injections of a bolus of the preparation or may be made more continuous by intravenous or intraperitoneal administration from a reservoir which is external (e.g., an IV bag) or internal (e.g., a bioerodable implant, a bioartificial organ, a biocompatible capsule of Meteorin production cells, or a colony of implanted Meteorin production cells). See, e.g., U.S. Pat. Nos. 4,407,957, 5,798,113, and 5,800,828, each incorporated herein by reference.

Localised delivery may be by such means as delivery via a catheter to one or more arteries. In one embodiment of the present invention localised delivery comprises delivery using encapsulated cells (as described in the section “biocompatible capsule”). A further type of localised delivery comprises local delivery of gene therapy vectors, which are normally injected.

In a preferred embodiment of the present invention the administration is parenteral injection, preferably subcutaneous injection or intrathecal injection.

Whilst it is possible for the compounds of the present invention to be administered as the raw chemical, it is preferred to present them in the form of a pharmaceutical formulation. The pharmaceutical formulations may be prepared by conventional techniques, e.g. as described in Remington: The Science and Practice of Pharmacy 2005, Lippincott, Williams & Wilkins.

The term “pharmaceutically acceptable carrier” means one or more organic or inorganic ingredients, natural or synthetic, with which Meteorin polypeptide is combined to facilitate its application. A suitable carrier includes sterile saline although other aqueous and non-aqueous isotonic sterile solutions and sterile suspensions known to be pharmaceutically acceptable are known to those of ordinary skill in the art.

The compounds of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. A recombinant Meteorin protein is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Where slow-release administration of a Meteorin polypeptide is desired in a formulation with release characteristics suitable for the treatment of any disease or disorder requiring administration of a Meteorin polypeptide, microencapsulation of a Meteorin polypeptide is contemplated. Microencapsulation of recombinant proteins for sustained release has been successfully performed with human growth hormone (rhGH), interferon-(rhIFN-), interleukin-2, and MN rgp120. Johnson et al. (1996); Yasuda (1993); Hora et al. (1990); Cleland, (1995), pp. 439-462; WO 97/03692, WO 96/40072, WO 96/07399; and U.S. Pat. No. 5,654,010.

The slow-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

In one embodiment of the present invention a composition comprising Meteorin is contemplated. The composition may comprise an isolated polypeptide as described herein, an isolated nucleic acid as described herein, a Meteorin encoding expression vector as described herein, a cell line expressing Meteorin as described herein or a biocompatible capsule secreting Meteorin as described herein.

### Dosages

Various dosing regimens for systemic administration are contemplated. In one embodiment, methods of administering to a subject a formulation comprising a Meteorin polypeptide include administering Meteorin at a dosage of between 1 μg/kg and 10,000 μg/kg body weight of the subject, per dose. In another embodiment, the dosage is between 1 μg/kg and 7,500 μg/kg body weight of the subject, per dose. In a further embodiment, the dosage is between 1 μg/kg and 5,000 μg/kg body weight of the subject, per dose. In a different embodiment, the dosage is between 1 μg/kg and 2,000 μg/kg body weight of the subject, per dose. In yet another embodiment, the dosage is between 1 μg/kg and 1,000 μg/kg body weight of the subject, per dose. In yet another embodiment, the dosage is between 1 μg/kg and 700 μg/kg body weight of the subject, per dose. In a more preferable embodiment, the dosage is between 5 μg/kg and 500 μg/kg body weight of the subject, per dose. In a most preferable embodiment, the dosage is between 10 μg/kg and 100 μg/kg body weight of the subject, per dose. In a preferred embodiment the subject to be treated is human.

Guidance as to particular dosages and methods of delivery is provided in the literature; see, for example, WO 02/78730 and WO 07/100898. Guidance to the calculation of the human equivalent dosages based on dosages used in animal experiments is provided in Reagan-Shaw et al., FASEB J, 22, 659-661 (2007).

The dose administered must be carefully adjusted to the age, weight and condition of the individual being treated, as well as the route of administration, dosage form and regimen, and the result desired, and the exact dosage should be determined by the practitioner.

In one embodiment of the present invention the administration is repeated daily. In another embodiment the administration is repeated at least 1-3 times weekly, such as 2-5 times weekly, such as 3-6 times weekly.

In one embodiment, the administration is repeated once a day, once every two days, once every three days, once every four days, once every five days, once every six days, or once every 7 days. In a preferred embodiment, the administration is repeated once every two days.

In one embodiment, the present invention provides treatment of chemotherapy-induced neuropathic pain. Thus, in one embodiment, the administration is initiated after onset of symptoms of neuropathic pain.

In one embodiment the administration of said polypeptide is initiated after initiation of chemotherapy treatment such as 1 day after, such as 2 days after, such as 3 days after, such as 4 days after, such as 5 days after, such as 8 days after, such as 12 days after initiation of chemotherapy treatment. In another embodiment administration of said polypeptide is initiated after initiation of chemotherapy treatment, such as 1 week after, such as 2 weeks after, such as 3 weeks after initiation of chemotherapy treatment.

In one embodiment, the present invention provides prevention of chemotherapy-induced neuropathic pain. Thus, in one embodiment, the neurotrophic polypeptide is administered prior to, simultaneously, or intermittently with chemotherapy treatment.

In one embodiment, the administration is initiated prior to initiation of chemotherapy treatment.

In one embodiment, the administration is initiated at least one day prior to initiation of chemotherapy treatment, such as at least two days prior to initiation of chemotherapy treatment, for example at least three days prior to initiation of chemotherapy treatment, such as at least 4 days, at least 5 days, at least 6 days, or at least one week prior to initiation of chemotherapy treatment.

In one embodiment, the neurotrophic polypeptide is administered on the same day as initiation of chemotherapy treatment, or at least one day prior to initiation of chemotherapy treatment, such as at least two days prior to initiation of chemotherapy treatment, for example at least three days prior to initiation of chemotherapy treatment, such as at least 4 days, at least 5 days, at least one week prior to initiation of chemotherapy treatment.

Chemotherapeutic treatment is often composed of multiple administrations of an anticancer agent at a given interval, such as once a week, once every two weeks, such as once every three weeks, such as once a month. In one embodiment, the neurotrophic polypeptide is administered in conjunction with each administration of the anticancer agent, such as administered prior to, simultaneously, or intermittently with each occurrence of administration of the anticancer agent.

In other embodiments, Meteorin is administered at relatively long dosage intervals. A relatively long dosage interval is intended to include at least 2 days between dosages, such as at least 3 days between dosages, for example 2 dosages per week. More preferably the long dosages intervals are at least one week, such as at least 2 weeks, more preferably at least 3 weeks, such as at least 4 weeks, or at least one month.

By a relatively long dosage interval is intended at least 2 days between dosages, such as at least 3 days between dosages, for example 2 dosages per week. More preferably the long dosages interval is at least one week, such as at least 2 weeks, more preferably at least 3 weeks, such as at least 4 weeks, or at least one month.

Expressed in a different way the dosage intervals are so long that following one dosage of Meteorin polypeptide, the polypeptide is no longer detectable in the serum of the subject to be treated when the next dosage is administered. In another embodiment the blood serum level is below 10 ng/ml, such as below 5 ng/ml, more preferably below 1 ng/mL, such as below 0.5 ng/ml, for example below 0.1 ng/mL.

In some embodiments, the long dosage range is preceded by more frequent initial administration of Meteorin, e.g., twice daily, daily, once every two days, once every three days, or once every four days. This initial dosing schedule may be maintained e.g., for 2, 3, 4, 5, 6, 7, 9, 11, 14, 21 days, or more. After completion of this dosing schedule, Meteorin can be administered less frequently, e.g., as described above.

Thus, in one aspect, the invention relates to a method of treating neuropathic pain in a human subject in need thereof comprising administering to the subject a therapeutically effective amount of a neurotrophic polypeptide comprising an amino acid sequence having at least 70% identity to the amino acid sequence of SEQ ID NO: 3. wherein said administration is three times per week or more infrequently.

Preferably, the administration is weekly or more infrequent administration. Even more preferably the administration is bi-weekly or more infrequent administration.

Expressed in a different way the dosage intervals are so long that following one dosage of Meteorin polypeptide, the polypeptide is no longer detectable in the serum of the subject to be treated when the next dosage is administered. In another embodiment the blood serum level is below 10 ng/ml, such as below 5 ng/ml, more preferably below 1 ng/ml, such as below 0.5 ng/ml, for example below 0.1 ng/ml.

In some embodiments, the initial administration of Meteorin is, e.g., twice daily, daily, once every two days, once every three days, or once every four days. This dosing schedule may be maintained e.g., for 2, 3, 4, 5, 6, 7, 9, 11, 14, 21 days, or more. After completion of this dosing schedule, Meteorin can be administered less frequently, e.g., as described above.

### Meteorin

The present invention relates to the use of polypeptides being identified as Meteorin protein and polynucleotides encoding said protein, in the treatment of chemotherapy-induced neuropathic pain. The delivery is in one embodiment contemplated to be by use of a capsule for delivery of a secreted biologically active Meteorin and/or a homologue thereof to a subject. The Meteorin protein has been identified in human beings (SEQ ID NO: 2), mouse (SEQ ID NO: 5), and rat (SEQ ID NO: 8) and a variety of other species.

Human Meteorin exists as a 293 amino acid precursor, which can be processed to give rise to at least one biologically active peptide. Meteorin is expressed at high levels in the nervous system and the eye, and in particular subregions of the brain. The mouse (SEQ ID NO: 5) and rat (SEQ ID NO: 8) Meteorin precursors consist of 291 amino acids, and the % sequence identities with the human Meteorin protein (SEQ ID NO: 2) are 80.3 and 80.2, respectively (See FIG. 8).

Human Meteorin contains an N-terminal signal peptide sequence of 23 amino acids, which is cleaved at the sequence motif ARA-GY. This signal peptide cleavage site is predicted by the SignalP method. The N-terminal of mouse Meteorin has been verified by N-terminal sequencing (Jørgensen et al., 2009).

Table 1 shows the % sequence identity between full length human Meteorin versus mouse and rat sequences. See alignment in FIG. 8a.

| Sequence | % sequence identity |
|---|---|
| Human | — |
| Mouse | 80.3 |
| Rat | 80.2 |

Table 2 shows the % sequence identity between human Meteorin versus mouse and rat sequences after removal of N-terminal signal peptide. See alignment in FIG. 8b.

| Sequence | % sequence identity |
|---|---|
| Human | — |
| Mouse | 81.9 |
| Rat | 79.6 |

Based on the fully conserved residues, a consensus sequence for mature Meteorin can be derived (SEQ ID NO: 11, FIG. 8c), wherein X is independently selected from any of the 21 naturally occurring amino acid encoded by DNA. In a preferred embodiment a variant Meteorin comprises the consensus sequence.

One biological function of Meteorin is the ability to induce neurite outgrowth in dissociated dorsal root ganglia (DRG) cultures as described in Jørgensen et al. (2009) and Nishino et al. (2004).

Due to the high conservation of the cysteines, it is expected that these residues play an important role in the secondary and tertiary structure of the bioactive protein. One or more of the cysteines may participate in the formation of intra- and/or intermolecular disulphide bridges.

### Meteorin Polypeptides

In addition to full-length Meteorin, substantially full-length Meteorin, and to pro-Meteorin, the present invention provides for biologically active variants of the polypeptides. A Meteorin polypeptide or fragment is biologically active if it exhibits a biological activity of naturally occurring Meteorin as described herein, such as being neurotrophic. It is to be understood that the invention relates to Meteorin as herein defined.

The invention relates to an isolated polypeptide molecule for use in a method of treatment of allodynia, hyperalgesia and/or spontaneous pain, said polypeptide comprising an amino acid sequence selected from the group consisting of:
a) the amino acid sequence selected from the group consisting of SEQ ID NO: 3, 6 and 9;
b) a biologically active sequence variant of the amino acid sequence selected from the group consisting of SEQ ID NO: 3, 6 and 9, wherein the variant has at least 70% sequence identity to said SEQ ID NO; and
c) a biologically active fragment of at least 50 contiguous amino acids of any of a) or b) wherein the fragment is at least 70% identical to said SEQ ID NO.

In one embodiment the invention relates to an isolated polypeptide selected from the group consisting of:
i) AA₃₀-AA₂₈₈ of SEQ ID NO: 2, and polypeptides having from one to five extra amino acids from the native sequence in one or both ends, up to AA₂₅-AA₂₉₃ of SEQ ID NO: 2;
ii) AA₂₈-AA₂₈₆ Of SEQ ID NO: 8 and polypeptides having from one to five extra amino acids from the native sequence in one or both ends, up to AA₂₃-AA₂₉₁ of SEQ ID NO: 8;
iii) AA₃₁-AA₂₈₉ of SEQ ID NO: 5 and polypeptides having from one to five extra amino acids from the native sequence in one or both ends, up to AA₂₆-AA₂₉₄ of SEQ ID NO: 5; and
iv) variants of said polypeptides, wherein any amino acid specified in the chosen sequence is changed to a different amino acid, provided that no more than 20 of the amino acid residues in the sequence are so changed.

Biological activity preferably is neurotrophic activity. Neurotrophically active variants may be defined with reference to one or more of the other in vitro and/or in vivo neurotrophic assays described above in WO 2005/095450, in particular the DRG assay.

A preferred biological activity is the ability to elicit substantially the same response as in the DRG assay described in Jørgensen et al. (2009). In this assay DRG cells are grown in the presence of full length human Meteorin coding sequence (SEQ ID NO: 3). By substantially the same response in the DRG assay is intended that the neurite outgrowth from DRG cells is at least 20% of the number obtained in the DRG assay described in Jørgensen et al. (2009), more preferably at least 30%, more preferably at least 40%, more preferably at least 50%, more preferably at least 60%, more preferably at least 70%, more preferably at least 75%, more preferably at least 80%, more preferably at least 85%, more preferably at least 90%. The biological activity of a fragment or variant of Meteorin may also be higher than that of the naturally occurring Meteorin (SEQ ID NO: 3).

Variants can differ from naturally occurring Meteorin in amino acid sequence or in ways that do not involve sequence, or in both ways. Variants in amino acid sequence (“sequence variants”) are produced when one or more amino acids in naturally occurring Meteorin is substituted with a different natural amino acid, an amino acid derivative or non-native amino acid. Particularly preferred variants include naturally occurring Meteorin, or biologically active fragments of naturally occurring Meteorin, whose sequences differ from the wild type sequence by one or more conservative and/or semi-conservative amino acid substitutions, which typically have minimal influence on the secondary and tertiary structure and hydrophobic nature of the protein or peptide.

Variants may also have sequences, which differ by one or more non-conservative amino acid substitutions, deletions or insertions, which do not abolish the Meteorin biological activity. The Clustal W alignment in FIG. 8 can be used to predict which amino acid residues can be substituted without substantially affecting the biological activity of the protein. In a preferred embodiment a variant Meteorin sequence comprises the consensus sequence having SEQ ID NO: 11.

Substitutions within the following group (Clustal W, ‘strong’ conservation group) are to be regarded as conservative substitutions within the meaning of the present invention -S,T,A; N,E,Q,K; N,H,Q,K; N,D,E,Q; Q,H,R,K; M,I,L,V; M,I,L,F; H,Y; F,Y,W.

Substitutions within the following group (Clustal W, ‘weak’ conservation group) are to be regarded as semi-conservative substitutions within the meaning of the present invention -C,S,A; A,T,V; S,A,G; S,T,N,K; S,T,P,A; S,G,N,D; S,N,D,E,Q,K; N,D,E,Q,H,K; N, E,Q,H,R,K; V,L,I,M; H,F,Y.

Other variants within the invention are those with modifications which increase peptide stability. Such variants may contain, for example, one or more nonpeptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D-amino acids instead of L-amino acids into the polypeptide may increase its resistance to proteases. See, e. g., U.S. Pat. No. 5,219,990. Splice variants are specifically included in the invention.

When the result of a given substitution cannot be predicted with certainty, the derivatives may be readily assayed according to the methods disclosed herein to determine the presence or absence of neurotrophic activity, preferably using the DRG assay described in Jørgensen et al., Characterization of meteorin-An evolutionary conserved neurotrophic factor, J Mol Neurosci 2009 Sep; 39 (1-2): 104-116.

In one embodiment, the polypeptide is a naturally occurring allelic variant of the sequence selected from the group consisting of SEQ ID NO: 3, 6 and 9. This polypeptide may comprise an amino acid sequence that is the translation of a nucleic acid sequence differing by a single nucleotide from a nucleic acid sequence selected from the group consisting of SEQ ID NO: 1, 4 and 7.

A variant polypeptide as described herein, in one embodiment comprises a polypeptide wherein any amino acid specified in the chosen sequence is changed to provide a conservative substitution.

Variants within the scope of the invention in one embodiment include proteins and peptides with amino acid sequences having at least 70 percent identity with human, murine or rat Meteorin (SEQ ID NO: 3, 6, and 9). More preferably the sequence identity is at least 75%, more preferably at least 80%, more preferably at least 85%, more preferably at least 90%, more preferably at least 95%, more preferably at least 98%.

In a preferred embodiment the sequence identity of the variant Meteorin is determined with reference to a human Meteorin polypeptide (SEQ ID NO: 3).

In one embodiment, the variants include proteins comprising an amino acid sequence having at least 70% sequence identity to SEQ ID NO: 3, more preferably at least 75%, more preferably at least 80%, more preferably at least 85%, more preferably at least 90%, more preferably at least 95%, more preferably at least 98%.

In one embodiment, preferred variants include proteins comprising an amino acid sequence having at least 70% sequence identity to SEQ ID NO: 6, more preferably at least 75%, more preferably at least 80%, more preferably at least 85%, more preferably at least 90%, more preferably at least 95%, more preferably at least 98%.

In one embodiment, preferred variants include proteins comprising an amino acid sequence having at least 70% sequence identity to SEQ ID NO: 9, more preferably at least 75%, more preferably at least 80%, more preferably at least 85%, more preferably at least 90%, more preferably at least 95%, more preferably at least 98%.

The neurotrophic polypeptide preferably has at least 85% sequence identity to the amino acid sequence of SEQ ID NO: 3, more preferably at least 90%, more preferably at least 95%, more preferably at least 98%.

In one embodiment the neurotrophic polypeptide comprises the consensus sequence of SEQ ID NO: 11.

Preferably the neurotrophic polypeptide has cysteine residues at positions 7, 28, 59, 95, 148, 151, 161, 219, 243, and 265 relative to the amino acid sequence of SEQ ID NO: 3.

In one embodiment, preferred variants of Meteorin include proteins comprising 50-270 amino acids, more preferably 75-270 amino acids, more preferably 90-270 amino acids, more preferably 100-270 amino acids, more preferably 125-270 amino acids, more preferably 150-270 amino acids, more preferably 175-270 amino acids, more preferably 200-270 amino acids, more preferably 225-270 amino acids, more preferably 250-270 amino acids.

In one embodiment, a variant Meteorin at corresponding positions comprises the residues marked in FIG. 8 as fully conserved (*), more preferably a variant Meteorin also comprises at corresponding positions the residues marked in FIG. 8 as strongly conserved (: strongly conserved groups include: S,T,A; N,E,Q,K; N,H,Q,K; N,D,E,Q; Q,H,R,K; M,I,L,V; M,I,L,F; H,Y; F,Y,W), more preferably a variant Meteorin also comprises at corresponding positions the residues marked in FIG. 8 as less conserved (. less conserved groups include: C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y). In particular, it is contemplated that the conserved cysteines must be located at corresponding positions in a variant Meteorin. Thus in one embodiment, a variant Meteorin sequence has cysteine residues at positions 7, 28, 59, 95, 148, 151, 161, 219, 243, and 265 relative to the amino acid sequence of SEQ ID NO: 3.

In one embodiment the neurotrophic polypeptide comprises the consensus sequence of SEQ ID NO:11. The consensus sequence comprises the amino acid residues conserved in human, mouse and rat Meteorin as shown in FIG. 8. Preferably the neurotrophic polypeptide has cysteine residues at positions 7, 28, 59, 95, 148, 151, 161, 219, 243, and 265 relative to the amino acid sequence of SEQ ID NO: 3.

Non-sequence modifications may include, for example, in vivo or in vitro chemical derivatisation of portions of naturally occurring Meteorin, as well as acetylation, methylation, phosphorylation, carboxylation, PEG-ylation, or glycosylation. Just as it is possible to replace substituents of the protein, it is also possible to substitute functional groups, which are bound to the protein with groups characterized by similar features. Such modifications do not alter primary sequence. These will initially be conservative, i.e., the replacement group will have approximately the same size, shape, hydrophobicity and charge as the original group.

Many amino acids, including the terminal amino acids, may be modified in a given polypeptide, either by natural processes such as glycosylation and other posttranslational modifications, or by chemical modification techniques which are well known in the art. Among the known modifications which may be present in polypeptides of the present invention are, to name an illustrative few, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance, Creighton (1993). Many detailed reviews are available on this subject, such as, for example, those provided by Wold, F. (1983); Seifter et al. (1990) and Rattan et al. (1992).

In addition, the protein may comprise a protein tag to allow subsequent purification and optionally removal of the tag using an endopeptidase. The tag may also comprise a protease cleavage site to facilitate subsequent removal of the tag. Non-limiting examples of affinity tags include a polyhis tag, a GST tag, a HA tag, a Flag tag, a C-myc tag, a HSV tag, a V5 tag, a maltose binding protein tag, a cellulose binding domain tag. Preferably for production and purification, the tag is a polyhis tag. Preferably, the tag is in the C-terminal portion of the protein.

The native signal sequence of Meteorin may also be replaced in order to increase secretion of the protein in recombinant production in other mammalian cell types.

Modifications can occur anywhere in a polypeptide, including the peptide backbone, the amino acid sidechains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well.

The modifications that occur in a polypeptide often will be a function of how it is made. For polypeptides made by expressing a cloned gene in a host, for instance, the nature and extent of the modifications in large part will be determined by the host cell's posttranslational modification capacity and the modification signals present in the polypeptide amino acid sequence. For instance, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation, inter alia. Similar considerations apply to other modifications.

It will be appreciated that the same type of modification may be present to the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

### Meteorin Nucleotide Sequences

The invention provides medical use of genomic DNA and cDNA coding for Meteorin, including for example the human cDNA nucleotide sequence (SEQ ID NO: 1 and 10), the mouse cDNA sequences (SEQ ID NO: 4) and rat cDNA sequences (SEQ ID NO: 7).

Variants of these sequences are also included within the scope of the present invention.

The invention relates to an isolated nucleic acid molecule for use in a method of treatment and/or prevention of chemotherapy-induced neuropathic pain, said nucleic acid molecule comprising a nucleic acid sequence encoding a polypeptide, said polypeptide comprising an amino acid sequence selected from the group consisting of:
i. The amino acid sequence of SEQ ID NO: 3;
ii. A biologically active sequence variant of the amino acid sequence of SEQ ID NO: 3, wherein the variant has at least 70% sequence identity to SEQ ID NO: 3; and
iii. A biologically active fragment of at least 50 contiguous amino acids of i) or ii) wherein the fragment is at least 70% identical to SEQ ID NO: 3.

In one embodiment the invention relates to an isolated nucleic acid molecule for use in a method of treatment and/or prevention of chemotherapy-induced neuropathic pain encoding a polypeptide, said polypeptide comprising an amino acid sequence selected from the group consisting of:
i) AA₃₀-AA₂₈₈ of SEQ ID NO: 2, and polypeptides having from one to five extra amino acids from the native sequence in one or both ends, up to AA₂₅-AA₂₉₃ of SEQ ID NO: 2;
ii) AA₂₈-AA₂₈₆ Of SEQ ID NO: 8 and polypeptides having from one to five extra amino acids from the native sequence in one or both ends, up to AA₂₃-AA₂₉₁ of SEQ ID NO: 8;
iii) AA₃₁-AA₂₈₉ of SEQ ID NO: 5 and polypeptides having from one to five extra amino acids from the native sequence in one or both ends, up to AA₂₆-AA₂₉₄ of SEQ ID NO: 5; and
iv) variants of said polypeptides, wherein any amino acid specified in the chosen sequence is changed to a different amino acid, provided that no more than 20 of the amino acid residues in the sequence are so changed.

The nucleic acid molecule may comprise the nucleotide sequence of a naturally occurring allelic nucleic acid variant.

The nucleic acid molecule of the invention may encode a variant polypeptide, wherein the variant polypeptide has the polypeptide sequence of a naturally occurring polypeptide variant.

In one embodiment the nucleic acid molecule differs by a single nucleotide from a nucleic acid sequence selected from the group consisting of SEQ ID NO: 1, 4, 7 and 10.

Preferably the encoded polypeptide has at least 60% sequence identity to a sequence selected from the group consisting of SEQ ID NO: 3 preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID NOs. Said sequences constitute human Meteorin.

In a preferred embodiment, the encoded polypeptide comprises the consensus sequence having SEQ ID NO: 11.

In a preferred embodiment the encoded polypeptide has at least 70% sequence identity to SEQ ID NO: 3, more preferably at least 75%, more preferably at least 80%, more preferably at least 95%, more preferably at least 98%, more preferably wherein said polypeptide has the sequence of SEQ ID NO: 3.

In one aspect the nucleic acid molecule comprises a nucleotide sequence selected from the group consisting of
a) the nucleotide sequence selected from the group consisting of SEQ ID NO: 1, 4, 7 and 10;
b) a nucleotide sequence having at least 70% sequence identity to a nucleotide sequence selected from the group consisting of SEQ ID NO: 1, 4, 7 and 10; and
c) a nucleic acid sequence of at least 150 contiguous nucleotides of a sequence selected from the group consisting of SEQ ID NO: 1, 4, 7 and 10;

In one embodiment, the isolated polynucleotide of the invention has at least 60, more preferably at least 65%, more preferably at least 70%, more preferably at least 75%, more preferably at least 80%, preferably at least 85%, more preferred at least 90%, more preferred at least 95%, more preferred at least 98% sequence identity to the polynucleotide sequence presented as SEQ ID NO: 1.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence presented as SEQ ID NO: 10.

In one embodiment, preferred isolated polynucleotide variants of the invention comprises 150-900 nucleic acids, more preferably 175-900 nucleic acids, more preferably 200-900 nucleic acids, more preferably 225-900 nucleic acids, more preferably 250-900 nucleic acids, more preferably 300-900 nucleic acids, more preferably 350-900 nucleic acids, more preferably 400-900 nucleic acids, more preferably 450-900 nucleic acids, more preferably 500-900 nucleic acids, more preferably 550-900 nucleic acids, more preferably 600-900 nucleic acids, more preferably 650-900 nucleic acids, more preferably 700-900 nucleic acids, more preferably 750-900 nucleic acids, more preferably 800-900 nucleic acids, more preferably 850-900 nucleic acids.

A preferred group of isolated polynucleotides include SEQ ID NO: 1 and 10, which are human Meteorin cDNA sequences. Generally the cDNA sequence is much shorter than the genomic sequences are more easily inserted into an appropriate expression vector and transduced/fected into a production cell or a human cell in vivo or ex vivo.

In addition, the nucleotide sequences of the invention include sequences, which are derivatives of these sequences. The invention also includes vectors, liposomes and other carrier vehicles, which encompass one of these sequences or a derivative of one of these sequences. The invention also includes proteins transcribed and translated from Meteorin cDNA, preferably human Meteorin cDNA, including but not limited to human Meteorin and derivatives and variants.

Codon optimised nucleic acid molecules for enhanced expression in selected host cells, including but not limited to E. coli, yeast species, Chinese Hamster, Baby Hamster, insect, fungus, and human are also contemplated.

Variant nucleic acids can be made by state of the art mutagenesis methods. Methods for shuffling coding sequences from human with those of mouse, rat or chimpanzee are also contemplated.

Variant nucleic acids made by exchanging amino acids present in human Meteorin with the amino acid present in mouse or rat Meteorin at the corresponding position, should this amino acid be different from the one present in human Meteorin.

### Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV. A further group of suitable retroviruses includes the group consisting of HIV, SIV, FIV, EAIV, CIV. Another group of preferred virus vectors includes the group consisting of alphavirus, adenovirus, adeno associated virus, baculovirus, HSV, coronavirus, Bovine papilloma virus, Mo-MLV, preferably adeno associated virus.

Preferred viruses for treatment of disorders of the nervous system are lentiviruses and adeno-associated viruses. Both types of viruses can integrate into the genome without cell divisions, and both types have been tested in pre-clinical animal studies for indications of the nervous system, in particular the central nervous system.

Methods for preparation of AAV are described in the art, e.g. U.S. Pat. Nos. 5,677,158, 6,309,634 and 6,683,058 describe examples of delivery of AAV to the central nervous system.

Preferably, a lentivirus vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome. Retroviruses can be used to target stem cells of the nervous system as very few cell divisions take place in other cells of the nervous system (in particular the CNS).

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous

DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the y signal and expressing the gag/pol genes and the env gene from separate transcriptional units. In addition, in some 2. and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

### Expression Vectors

Construction of vectors for recombinant expression of Meteorin polypeptides for use in the invention may be accomplished using conventional techniques which do not require detailed explanation to one of ordinary skill in the art. For review, however, those of ordinary skill may wish to consult Maniatis et al. (1982). Expression vectors may be used for generating producer cells for recombinant production of Meteorin polypeptides for medical use, and for generating therapeutic cells secreting Meteorin polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al. (1981), the method of Maxam, et al. (1980), or other suitable methods which will be known to those skilled in the art.

Size separation of cleaved fragments is performed using conventional gel electrophoresis as described, for example, by Maniatis, et al. (pp. 133-134,1982).

For generation of efficient expression vectors, these should contain regulatory sequences necessary for expression of the encoded gene in the correct reading frame. Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al. (1981); Corden et al (1980); and Breathnach and Chambon (1981)). For retroviruses, control elements involved in the replication of the retroviral genome reside in the long terminal repeat (LTR) (Weiss et al. (1982)). Moloney murine leukemia virus (MLV) and

Rous sarcoma virus (RSV) LTRs contain promoter and enhancer sequences (Jolly et al. (1983); Capecchi et al. (1991). Other potent promoters include those derived from cytomegalovirus (CMV) and other wild-type viral promoters.

Promoter and enhancer regions of a number of non-viral promoters have also been described (Schmidt et al. (1985); Rossi and deCrombrugghe, (1987)). Methods for maintaining and increasing expression of transgenes in quiescent cells include the use of promoters including collagen type I (1 and 2) (Prockop and Kivirikko (1984); Smith and Niles (1980); de Wet et al. (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, Elongation Factor 1 alpha promoter (EF1-alpha), RSV, CAG. Examples of inducible/repressible promoters include: Tet-On, Tet-Off, Rapamycin-inducible promoter, Mx1, Mo-MLV-LTR, progesterone, RU486.

A group of preferred promoters include CAG, CMV, human UbiC, JeT, SV40, RSV, Tet-regulatable promoter, Mo-MLV-LTR, Mx1, Mt1 and EF-1alpha.

In addition to using viral and non-viral promoters to drive transgene expression, an enhancer sequence may be used to increase the level of transgene expression. Enhancers can increase the transcriptional activity not only of their native gene but also of some foreign genes (Armelor (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al. (1981); Benoist and Chambon (1981), and Fromm and Berg (1982), all of which are incorporated by reference herein. This repeat sequence can increase the transcription of many different viral and cellular genes when it is present in series with various promoters (Moreau et al. (1981)).

Further expression enhancing sequences include but are not limited to Woodchuck hepatitis virus post-transcriptional regulation element, WPRE, SP163, CMV enhancer, and Chicken [beta]-globin insulator or other insulators.

### Cell Lines

In one aspect the invention relates to isolated host cells genetically modified with the vector according to the invention.

The invention also relates to cells suitable for biodelivery of Meteorin via naked or encapsulated cells, which are genetically modified to overexpress Meteorin, and which can be transplanted to the patient to deliver bioactive Meteorin polypeptide locally. Such cells may broadly be referred to as therapeutic cells.

For ex vivo gene therapy, the preferred group of cells includes neuronal cells, neuronal precursor cells, neuronal progenitor cells, neuronal stem cells, human glial stem cells, human precursor cells, stem cells and foetal cells.

For encapsulation the preferred cells include retinal pigmented epithelial cells, including ARPE-19 cells; human immortalised fibroblasts; and human immortalised astrocytes.

The ARPE-19 cell line is a superior platform cell line for encapsulated cell-based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long-life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic. Methods for culture and encapsulation of ARPE-19 cells are described in U.S. Pat. No. 6,361,771.

In another embodiment the therapeutic cell line is selected from the group consisting of: human fibroblast cell lines, human astrocyte cell lines, human mesencephalic cell line, and human endothelial cell line, preferably immortalised with TERT, SV40T or vmyc.

### Extracellular Matrix

The present invention further comprises culturing Meteorin producing cells in vitro on an extracellular matrix prior to implantation into the mammalian nervous system. The pre-adhesion of cells to microcarriers prior to implantation is designed to enhance the long-term viability of the transplanted cells and provide long term functional benefit.

Materials of which the extracellular matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances, or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of RPE cells. Thus, for example, the matrix material may be extracellular matrix or basement membrane material, which is produced and secreted by RPE cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, see, for example, Fisher Biotech Source 87-88, Fisher Scientific Co., 1987, pp. 72-75; Sigma Cell Culture Catalog, Sigma Chemical Co., St, Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### Examples

### Example 1: Pre-emptive treatment with Meteorin reverses PTX induced neuropathic pain

### Materials and Methods:

Female ICR/C57BI6J mice (n=8 per group) with an average weight of 23 g were divided into three groups; 1: paclitaxel (PTX) and Vehicle, 2: PTX and rmMeteorin (0.5 mg/kg), and 3: PTX and rmMeteorin (1.8 mg/kg). Subcutaneous (s.c) injection of either vehicle (Dulbecco's PBS) or rmMeteorin (0.5 mg/kg or 1.8 mg/kg) was administered every other day (D1, D3, D5, D7, and D9) using an insulin syringe (30G) as shown in FIG. 1. PTX in Kholepher-ethanol (1:1) was diluted in Dulbecco's-PBS and administered via intraperitoneal injection at a dosage of 4 mg/kg every other day (D2, D4, D6, and D8) for a total dose of 16 mg/kg. Mice were habituated for 2 h to clear acrylic behavioural chambers before beginning the experiment. The paw withdrawal threshold (PWT) was tested at baseline and then every or day using calibrated von Frey filaments until Day 57 as a surrogate marker of mechanical allodynia. At Day 24, 4 animals per treatment group were euthanized for histological staining (results summarized in example 2). Statistical analysis between groups was made using mixed-effects ANOVA. All data are represented as mean+/−SEM with p<0.05 considered significant.

### Results:

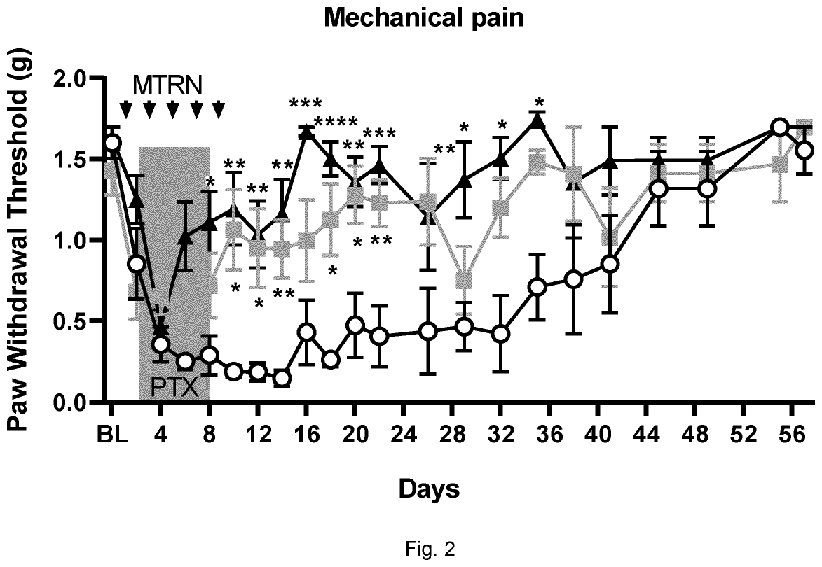

At Day 4 all mice developed robust mechanical allodynia induced by PTX treatment as shown in FIG. 2. With the continued intermittent administration of rmMeteorin an increase in the PWT for both the 0.5 mg/kg (grey squares) and 1.8 mg/kg (black triangles) was observed at Day 10 and Day 8 respectively. Thereafter, a full reversal of the PWT for the 0.5 and 1.8 mg/kg dose was obtained at Day 20 (P<0.001) and Day 16 (P<0.0001) respectively. This effect was essentially maintained throughout the duration of the experiment. However, from Day 32 onwards the PWT started to gradually increase towards baseline levels in Vehicle treated mice, the implication of which is that the Meteorin-mediated reversal of PWT in PTX mice only remained significant until Day 35.

### Conclusions:

Preventive treatment with repeated s.c. injections of rmMeteorin dose-dependently reversed paclitaxel-induced mechanical allodynia within days after initiation of dosing.

Moreover, although Meteorin treatment was completed by Day 8 this reversal continued thereby preventing any re-occurrence of neuropathic hypersensitivity.

### Example 2: Pre-Emptive Treatment with Meteorin Prevents Paclitaxel-Induced Immunohistochemical Changes in Hyperexcitability Markers within Dorsal Root Ganglia

### Materials and Methods:

Mice were anesthetized with isoflurane (4%) and euthanized by decapitation. Tissues were flash-frozen in O.C.T. on dry ice, and sections of DRG (20 μm) mounted onto SuperFrost Plus slides (Thermo Fisher Scientific, Waltham, MA). They were then fixed in ice-cold 10% formalin 15 min followed by incubation for 5 min in an increasing percentage of ethanol 50%,70%, 100%. Slides were then transferred to a blocking solution (10% Normal Goat Serum, 0.3% Triton-X 100 in 0.1 M phosphate buffer (PB) for 1 h at room temperature with gentle rocking/agitation. Sections were incubated in primary antibody (peripherin, glutamine synthetase, Connexin 43) diluted in blocking solution for 3 h at room temperature or 4° C. overnight. Sections were washed five times in 0.1 M PB and then incubated in secondary anti-body diluted in blocking solution containing DAPI for 1 h at room temperature. Sections were washed five times in 0.1 M PB, mounted onto glass slides, cover-slipped using Prolong Gold Antifade (Thermo Fisher Scientific, P36930), and sealed with nail polish. Images were taken using an Olympus FluoView 1200 confocal microscope). Analysis of immunohistochemical images obtained from 3-4 animals per treatment group was performed using Cellsens (Olympus).

### Results:

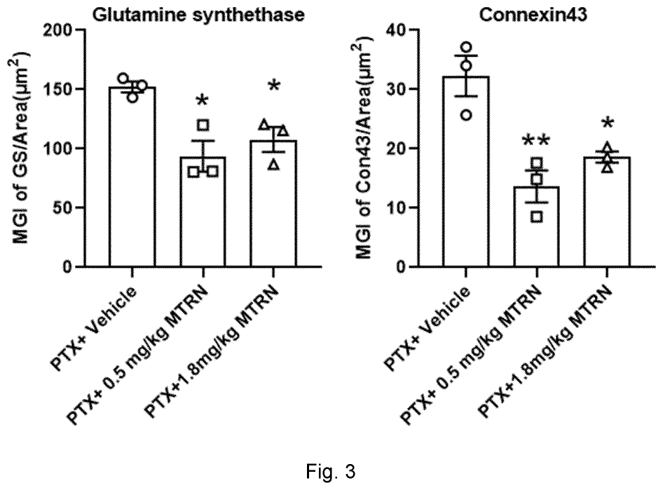

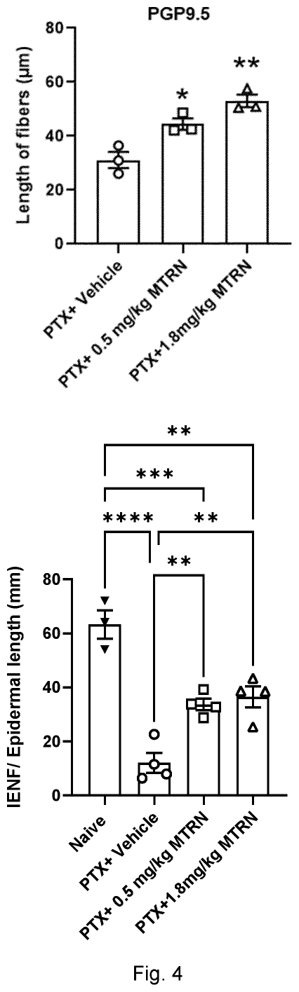

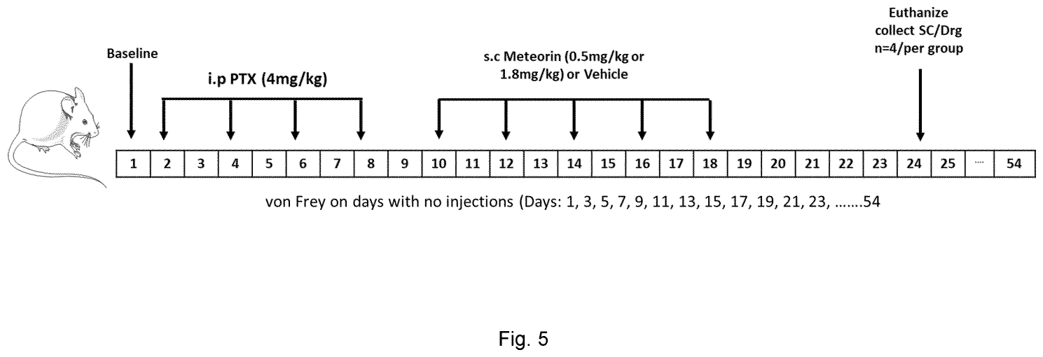

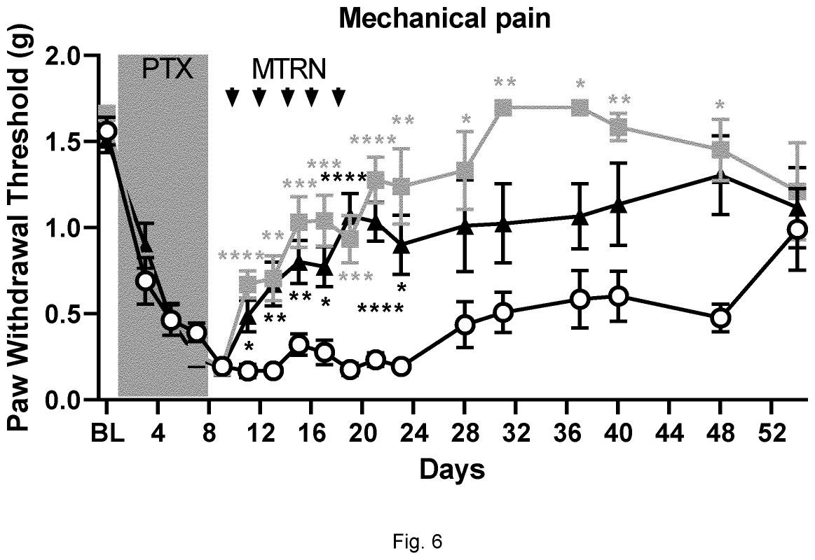

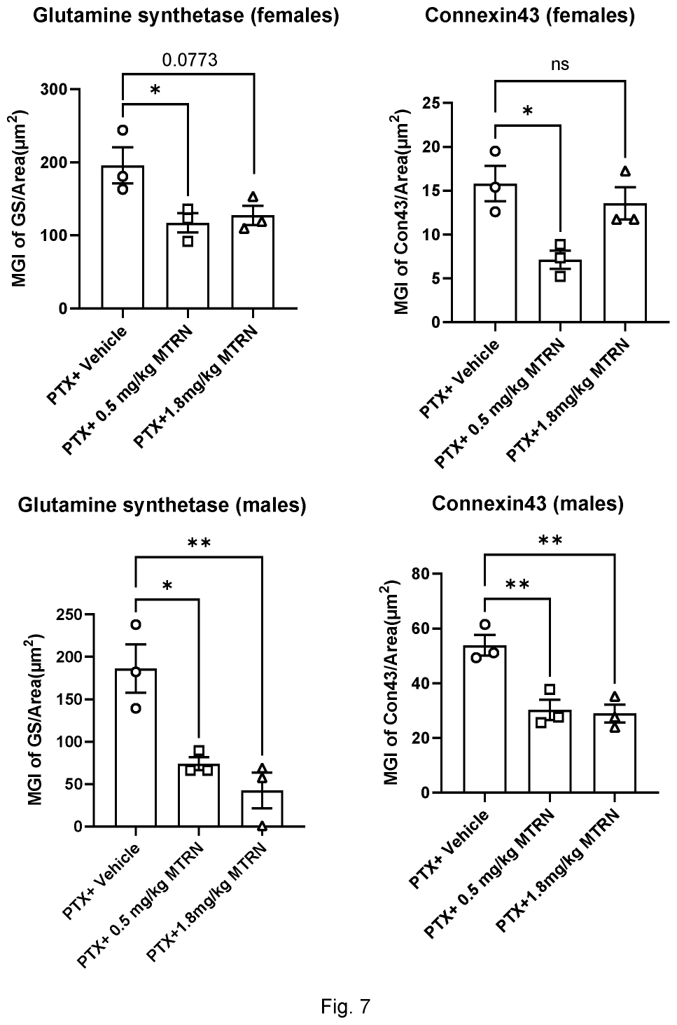

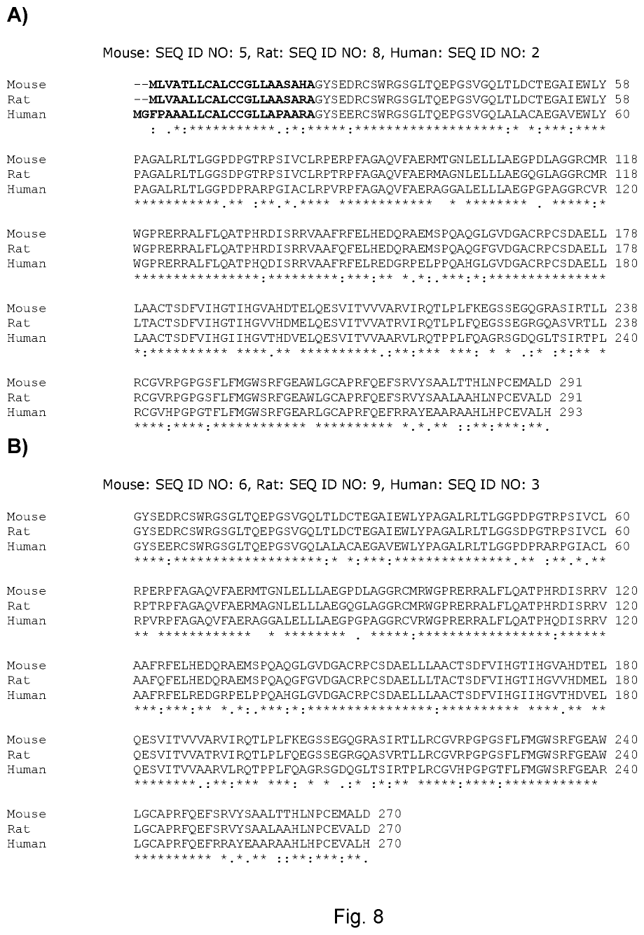

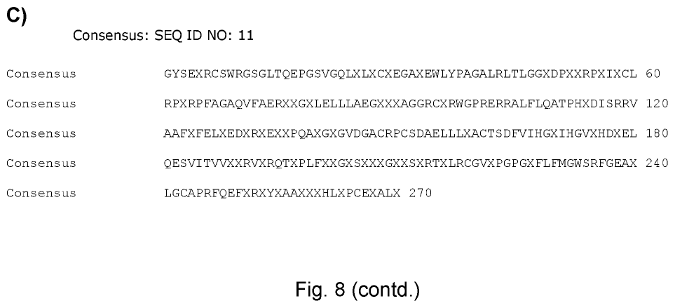

Increased connectivity between satellite glial cells and neuronal cell bodies after neuropathic injury contributes to increased electrical coupling and excitability within DRG tissue, which in turn manifests as signs of neuropathic pain. FIG. 3 indicates that paclitaxel(PTX)-mediated expression of the enzyme glutamine synthetase (GS) which is a specific marker of satellite glial cells was reduced by rmMeteorin treatment. Connexin 43 is a key gap junction protein that plays an important contributory role to hyperexcitability of DRG neurones after injury (Kim et al., 2016). FIG. 3 illustrates that PTX-induced Connexin 43 expression which encapsulated peripherin stained neuronal cell bodies within DRG tissue was prevented by pre-emptive treatment with rmMeteorin.

### Conclusions:

Glutamine synthethase and Connexin 43 are surrogate markers of PTX-induced hyperexcitability changes that occur within DRG tissue that are associated with behavioural neuropathic hypersensitivity. The reduction in expression of both proteins after repeated s.c. injections of rmMeteorin indicates that diminished neuronal-glia cell coupling is a potential pathophysiological mechanism targeted by rmMeteorin to mediate analgesia in CINP.

### Example 3: Pre-Emptive Treatment with Meteorin Prevents the Loss of Intraepidermal Nerve Fibres within the Skin of Paclitaxel-Treated Female Mice

### Materials and Methods:

Skin sections were postfixed in 10% formalin solution for 24 h followed by 30% sucrose solution for 48 h. 20 μm skin sections were cut using the cryostat followed by the antigen retrieval step using 0.15 mg/ml pepsin in 0.2M HCl. The sections were washed three times in 0.1M PB and transferred to primary antibody solution (PGP9.5) for immunohistochemical processing as described in Example 2 to facilitate staining of intra-epidermal nerve fibers (IENFs). Analysis of images using Cellsens software was performed as described in Example 2, Materials and methods.

### Results:

Treatment with paclitaxel in mice in addition to producing robust behavioural hyperalgesia results in a loss of IENFs from the skin of the mouse footpad (Singhmar et al., 2018). This reflects a corresponding loss of IENFs in some clinical neuropathic pain conditions. FIG. 4 shows that in mice treated with both 0.5 and 1.8 mg/kg doses of rmMeteorin, IENFs crossing the basal membrane of the epidermis were both longer and more intensely stained than in vehicle treated mice.

### Conclusions:

Pre-emptive treatment with repeated s.c. injections of rmMeteorin prevented the loss of IENFs innervating the hindpaw skin of paclitaxel-treated female mice, reflecting a disease-modifying effect of rmMeteorin in CINP.

### Example 4: Interventive Treatment with Meteorin Reverses Paclitaxel-Induced Neuropathic Pain

### Materials and Methods:

Adult ICR/C57BI6J mice (n=7-8 per group) were used in 2 identically designed experiments illustrated in FIG. 5, in which the first experiment used females (body weight 27 g), and the second experiment used males (body weight 34 g). PTX in Kholepher-ethanol (1:1) was diluted in Dulbecco's-PBS and administered via intraperitoneal injection at a dosage of 4 mg/kg every other day (D2, D4, D6, and D8) for a total dose of 16 mg/kg as shown in FIG. 5. On Day 10, the mice were divided into three groups; paclitaxel (PTX) and Vehicle, PTX and rmMeteorin (0.5 mg/kg), and PTX and rmMeteorin (1.8 mg/kg). Subcutaneous (s.c.) injection of either vehicle (Dulbecco's PBS) or rmMeteorin (0.5 mg/kg or 1.8 mg/kg) was administered every other day (Dayl0, D12, D14, D16, and D18) using an insulin syringe (30G). Mice were habituated for 2 h to clear acrylic behavioral chambers before beginning the experiment. Mechanical allodynia was tested every other day (days with no injections) using calibrated von Frey filaments. Mechanical allodynia was tested on the cohorts of mice until all the mice reached baseline. At Day 24, 4 animals per treatment group were euthanized for histological staining. Statistical analysis between groups was made using mixed-effects ANOVA. All data are represented as mean+/−SEM with p<0.05 considered significant.

### Results:

Data from the separate interventive experiments using female and male mice were combined for the purposes of analysis. After the first injection of PTX mice already started to develop hindpaw mechanical allodynia as shown in FIG. 6. After the fourth PTX injection the reduction in PWT reached a maximal value by Day 9. Following the first injection of rmMeteorin, both the 0.5 and 1.8 mg/kg treatment groups displayed a significant reversal in the PWT compared with Vehicle treatment. This reversal continued throughout the duration of rmMeteorin treatment and upon its cessation. Notably, the reversal of PTX-induced mechanical allodynia continued until Day 48 in rmMeteorin-treated mice.

### Conclusions:

Interventive treatment with repeated s.c. injections of both a low and high dose of rmMeteorin fully reversed paclitaxel-induced mechanical allodynia within days after initiation of dosing. Moreover, although rmMeteorin treatment was completed by Day 18 this reversal continued thereby preventing any re-occurrence of neuropathic hypersensitivity.

### Example 5: Interventive Treatment with Meteorin Reverses Paclitaxel-Induced Immunohistochemical Changes in Hyperexcitability Markers within Dorsal Root Ganglia

### Materials and Methods:

At Day 24 DRG tissue was obtained from female (n=9 total) and male (n=9 total) PTX mice previously treated with either s.c. Vehicle (n=3) or s.c. rmMeteorin (0.5 and 1.8 mg/kg, n=3 each group) and immunohistochemistry for peripherin (not shown), glutamine synthetase and Connexin 43 performed and analysed as described in Example 2.

### Results:

Increased connectivity between satellite glial cells and neuronal cell bodies after neuropathic injury contributes to increased electrical coupling and excitability within DRG tissue, which in turn manifests as signs of neuropathic pain. FIG. 7 indicates that paclitaxel-mediated expression of the enzyme glutamine synthetase (GS) which is a specific marker of satellite glial cells was reduced by rmMeteorin treatment in both female and male mice. Connexin 43 is a key gap junction protein that plays an important contributory role to hyperexcitability of DRG neurones after injury (Kim et al., 2016). FIG. 7 illustrates that PTX-induced Connexin 43 expression which encapsulated peripherin stained neuronal cell bodies within DRG tissue was reduced by treatment with rmMeteorin in both female and male mice.

### Conclusions:

Glutamine synthethase and Connexin 43 are surrogate markers of PTX-induced hyperexcitability changes that occur within DRG tissue that are associated with behavioural neuropathic hypersensitivity. The reduction in expression of both proteins after repeated interventive s.c. injections of rmMeteorin indicates that diminished neuronal-glia cell coupling is a potential pathophysiological mechanism targeted by rmMeteorin to mediate analgesia in CINP in both female and male mice.

### Sequence Overview

SEQ ID NO: 1: human Meteorin cDNA

SEQ ID NO: 2: human Meteorin full length amino acid sequence

SEQ ID NO: 3: human Meteorin amino acid sequence without signal peptide

SEQ ID NO: 4: mouse Meteorin cDNA

SEQ ID NO: 5: mouse Meteorin full length amino acid sequence

SEQ ID NO: 6: mouse Meteorin amino acid sequence without signal peptide

SEQ ID NO: 7: rat Meteorin cDNA

SEQ ID NO: 8: rat Meteorin full length amino acid sequence

SEQ ID NO: 9: rat Meteorin amino acid sequence without signal peptide

SEQ ID NO: 10: human codon optimized DNA sequence

SEQ ID NO: 11: mature Meteorin, consensus sequence

| Human Meteorin cDNA (1109 bp; CDS = 118-999) | |
|---|---|
| >gi\|34147349\|ref\|NM_024042.2\|Homo sapiens | |
| hypothetical protein MGC2601 (MGC2601), mRNA | |
| (SEQ ID NO: 1) | |
| GCTTCGCCGGGGCCGGGCGGCCGGCGCCCCCGGCTGCTCCCGCCGCCGCCCGGACCCGCGCCCCGCCGGG | |
| GCAGCGGTGGTGAGAGCCCCGACTCCCCGGACGCCGCCCGCCGTGCCATGGGGTTCCCGGCCGCGGCGCT | |
| GCTCTGCGCGCTGTGCTGCGGCCTCCTGGCCCCGGCTGCCCGCGCCGGCTACTCCGAGGAGCGCTGCAGC | |
| TGGAGGGGCAGCGGCCTCACCCAGGAGCCCGGCAGCGTGGGGCAGCTGGCCCTGGCCTGTGCGGAGGGCG | |
| CGGTTGAGTGGCTGTACCCGGCTGGGGCGCTGCGCCTGACCCTGGGCGGCCCCGATCCCAGAGCGCGGCC | |
| CGGCATCGCCTGTCTGCGGCCGGTGCGGCCCTTCGCGGGCGCCCAGGTCTTCGCGGAGCGCGCAGGGGGC | |
| GCCCTGGAGCTGCTGCTGGCCGAGGGCCCGGGCCCGGCAGGGGGCCGCTGCGTGCGCTGGGGTCCCCGCG | |
| AGCGCCGGGCCCTCTTCCTGCAGGCCACGCCGCACCAGGACATCAGCCGCCGCGTGGCCGCCTTCCGCTT | |
| TGAGCTGCGCGAGGACGGGCGCCCCGAGCTGCCCCCGCAGGCCCACGGTCTCGGCGTAGACGGTGCCTGC | |
| AGGCCCTGCAGCGACGCTGAGCTGCTCCTGGCCGCATGCACCAGCGACTTCGTAATTCACGGGATCATCC | |
| ATGGGGTCACCCATGACGTGGAGCTGCAGGAGTCTGTCATCACTGTGGTGGCCGCCCGTGTCCTCCGCCA | |
| GACACCGCCGCTGTTCCAGGCGGGGCGATCCGGGGACCAGGGGCTGACCTCCATTCGTACCCCACTGCGC | |
| TGTGGCGTCCACCCGGGCCCAGGCACCTTCCTCTTCATGGGCTGGAGCCGCTTTGGGGAGGCCCGGCTGG | |
| GCTGTGCCCCACGATTCCAGGAGTTCCGCCGTGCCTACGAGGCTGCCCGTGCTGCCCACCTCCACCCCTG | |
| CGAGGTGGCGCTGCACTGAGGGGCTGGGTGCTGGGGAGGGGCTGGTAGGAGGGAGGGTGGGCCCACTGCT | |
| TTGGAGGTGATGGGACTATCAATAAGAACTCTGTTCACGCAAAAAAAAAAAAAAAAAAA | |
| Human Meteorin full length amino acid sequence | |
| >IPI00031531.1 REFSEQ_NP:NP_076947 TREMBL:Q9UJH9 | |
| ENSEMBL:ENSP00000219542 Tax_Id = 9606 C380A1.2.1 | |
| (Novel protein) | |
| (SEQ ID NO: 2) | |
| MGFPAAALLC ALCCGLLAPA ARAGYSEERC SWRGSGLTQE PGSVGQLALA CAEGAVEWLY | |
| PAGALRLTLG GPDPRARPGI ACLRPVRPFA GAQVFAERAG GALELLLAEG PGPAGGRCVR | |
| WGPRERRALF LQATPHQDIS RRVAAFRFEL REDGRPELPP QAHGLGVDGA CRPCSDAELL | |
| LAACTSDFVI HGIIHGVTHD VELQESVITV VAARVLRQTP PLFQAGRSGD QGLTSIRTPL | |
| RCGVHPGPGT FLFMGWSRFG EARLGCAPRF QEFRRAYEAA RAAHLHPCEV ALH | |
| Human Meteorin, protein without signal peptide | |
| (SEQ ID NO: 3) | |
| GYSEERCSWR GSGLTQEPGS VGQLALACAE GAVEWLYPAG ALRLTLGGPD PRARPGIACL | |
| RPVRPFAGAQ VFAERAGGAL ELLLAEGPGP AGGRCVRWGP RERRALFLQA TPHQDISRRV | |
| AAFRFELRED GRPELPPQAH GLGVDGACRP CSDAELLLAA CTSDFVIHGI IHGVTHDVEL | |
| QESVITVVAA RVLRQTPPLF QAGRSGDQGL TSIRTPLRCG VHPGPGTELF MGWSRFGEAR | |
| LGCAPRFQEF RRAYEAARAA HLHPCEVALH | |
| Mouse Meteorin cDNA, 1363 bp, CDS 84..959 | |
| NM_133719. Mus musculus meteorin.[gi:56550040] | |
| (SEQ ID NO: 4) | |
| gggcagccgc gccgcgggct gctcgcgctg cggccccgac cctcccgggg cagcagtccg | |
| aggccccggc gcgtccccta accatgctgg tagccacgct tctttgcgcg ctctgttgcg | |
| gcctcctggc cgcgtccgct cacgctggct actcggaaga ccgctgcagc tggaggggca | |
| gcggtttgac ccaggagcct ggcagcgtgg ggcagctgac cctggactgt actgagggcg | |
| ctatcgagtg gctgtaccca gctggggcgc tgcgcctgac cctgggcggc cccgatccgg | |
| gcacacggcc cagcatcgtc tgtctgcgcc cagagcggcc cttcgctggt gcccaggtct | |
| tcgctgaacg tatgaccggc aatctagagt tgctactggc cgagggcccg gacctggctg | |
| ggggccgctg catgcgctgg ggtccccgcg agcgccgagc ccttttcctg caggccacac | |
| cacaccgcga catcagccgc agagttgctg ccttccgttt tgaactgcac gaggaccaac | |
| gtgcagaaat ccatgacaca gagctgcaag aatcagtcat cactgtggtg aggccctgca | |
| gtgatgccga gtctccccag gctcaaggtc ttggtgtgga tggtgcctgc gggaccatcc | |
| atggggtcgc gctcctcctg gctgcatgca ccagtgattt tgtgatccac gttgctcgtg | |
| tcatccgcca gacactgcca ctgttcaagg aagggagctc ggagggccaa ggccgggcct | |
| ccattcgtac cttgctgcgc tgtggtgtgc gtcctggccc aggctccttc ctcttcatgg | |
| gctggagccg atttggcgaa gcttggctgg gctgtgctcc ccgcttccaa gagttcagcc | |
| gtgtctattc agctgctctc acgacccatc tcaacccatg tgagatggca ctggactgag | |
| agacctggga gcaagccctg gatggacctt cttctggaga tggggtgttg gggagggtga | |
| tgggagggtg ggtgagaagg gtgtggctcg gatggcatcc tggtacccac agtgagctgg | |
| tagaatacta agtaatctgg accataccag ccactgtagt catggtcttc tgtggcaggc | |
| agcataccca gctctgtgcc tgcctcactt tgtctactct ccagtctgct gcccttctaa | |
| cccttcttag cctgctgacc agtgagctca tgttttcctc gaattccagg gtgctgctgg | |
| ggttcagagc aaccgtgccg tagtttggaa gacttgagct aattgttttt tttttgtttg | |
| tttttttgtt tgtttaaagg tggcctgggg ggggcggcaa aca | |
| Mouse Meteorin full length amino acid sequence | |
| ref\|NP_598480.1\|meteorin [Mus musculus] | |
| (SEQ ID NO: 5) | |
| MLVATLLCAL CCGLLAASAH AGYSEDRCSW RGSGLTQEPG SVGQLTLDCT EGAIEWLYPA | |
| GALRLTLGGP DPGTRPSIVC LRPERPFAGA QVFAERMTGN LELLLAEGPD LAGGRCMRWG | |
| PRERRALFLQ ATPHRDISRR VAAFRFELHE DQRAEMSPQA QGLGVDGACR PCSDAELLLA | |
| ACTSDFVIHG TIHGVAHDTE LQESVITVVV ARVIRQTLPL FKEGSSEGQG RASIRTLLRC | |
| GVRPGPGSFL FMGWSRFGEA WLGCAPRFQE FSRVYSAALT THLNPCEMAL D | |
| Mouse Meteorin protein without signal peptide | |
| (SEQ ID NO: 6) | |
| GYSEDRCSWR GSGLTQEPGS VGQLTLDCTE GAIEWLYPAG ALRLTLGGPD PGTRPSIVCL | |
| RPERPFAGAQ VFAERMTGNL ELLLAEGPDL AGGRCMRWGP RERRALFLQA TPHRDISRRV | |
| AAFRFELHED QRAEMSPQAQ GLGVDGACRP CSDAELLLAA CTSDFVIHGT IHGVAHDTEL | |
| QESVITVVVA RVIRQTLPLF KEGSSEGQGR ASIRTLLRCG VRPGPGSFLF MGWSRFGEAW | |
| LGCAPRFQEF SRVYSAALTT HLNPCEMALD | |
| Rat Meteorin cDNA (1026 bp; CDS = 1-876) | |
| >gi\|34870570\|ref\|XM_213261.2\| Rattus norvegicus | |
| similar to 1810034B16Rik protein | |
| (LOC287151), mRNA | |
| (SEQ ID NO: 7) | |
| ATGCTGGTAGCGGCGCTTCTCTGCGCGCTGTGCTGCGGCCTCTTGGCTGCGTCCGCTCGAGCTGGCTACT | |
| CCGAGGACCGCTGCAGCTGGAGGGGCAGCGGTTTGACCCAGGAACCTGGCAGCGTGGGGCAGCTGACCCT | |
| GGATTGTACTGAGGGTGCTATCGAGTGGCTGTATCCAGCTGGGGCGCTGCGCCTGACTCTAGGCGGCTCT | |
| GATCCGGGCACGCGGCCCAGCATCGTCTGTCTGCGCCCAACACGGCCCTTCGCTGGTGCCCAGGTCTTCG | |
| CTGAACGGATGGCCGGCAACCTAGAGTTGCTACTGGCCGAGGGCCAAGGCCTGGCTGGGGGCCGCTGCAT | |
| GCGCTGGGGTCCTCGCGAGCGCCGAGCCCTTTTCCTGCAGGCCACGCCACACCGGGACATCAGCCGCAGA | |
| GTTGCTGCCTTCCAATTTGAACTGCACGAGGACCAACGTGCAGAAATGTCTCCCCAGGCCCAAGGTTTTG | |
| GTGTGGATGGTGCCTGCAGGCCCTGCAGTGATGCCGAGCTCCTTCTGACTGCATGCACCAGTGACTTTGT | |
| GATCCATGGGACCATCCATGGGGTCGTCCATGACATGGAGCTGCAAGAATCAGTCATCACTGTGGTGGCC | |
| ACTCGTGTCATCCGCCAGACACTGCCACTGTTCCAGGAAGGGAGCTCGGAGGGCCGGGGCCAGGCCTCCG | |
| TTCGTACCTTGTTGCGCTGTGGTGTGCGTCCTGGCCCAGGCTCCTTCCTCTTCATGGGCTGGAGCCGATT | |
| TGGCGAAGCTTGGCTGGGCTGCGCTCCCCGCTTCCAAGAGTTCAGCCGTGTCTATTCAGCTGCTCTCGCG | |
| GCCCACCTCAACCCATGTGAGGTGGCACTGGACTGAGAGACCTGGGAGCAAGCCCTGGATGGATCTTCCT | |
| CTGGGGATGGGGTGTTGGGGAGGGGTGATAGGAGGGTGGGTGGGAAGGGTGTGGCTCAGATGGCATCCTG | |
| GTACCCACAGTGAGGTGGTAGAATACTAAATAACCTGGATCACACC | |
| Rat Meteorin full length amino acid sequence | |
| >IPI00369281.1\|REFSEQ_XP:XP_213261\|ENSEMBL: | |
| ENSRNOP00000026676 | |
| (SEQ ID NO: 8) | |
| MLVAALLCAL CCGLLAASAR AGYSEDRCSW RGSGLTQEPG SVGQLTLDCT EGAIEWLYPA | |
| GALRLTLGGS DPGTRPSIVC LRPTRPFAGA QVFAERMAGN LELLLAEGQG LAGGRCMRWG | |
| PRERRALFLQ ATPHRDISRR VAAFQFELHE DQRAEMSPQA QGFGVDGACR PCSDAELLLT | |
| ACTSDFVIHG TIHGVVHDME LQESVITVVA TRVIRQTLPL FQEGSSEGRG QASVRTLLRC | |
| GVRPGPGSFL FMGWSRFGEA WLGCAPREQE FSRVYSAALA AHLNPCEVAL D | |
| Rat Meteorin, protein without signal peptide | |
| (SEQ ID NO: 9) | |
| GYSEDRCSWR GSGLTQEPGS VGQLTLDCTE GAIEWLYPAG ALRLTLGGSD PGTRPSIVCL | |
| RPTRPFAGAQ VFAERMAGNL ELLLAEGQGL AGGRCMRWGP RERRALFLQA TPHRDISRRV | |
| AAFQFELHED QRAEMSPQAQ GFGVDGACRP CSDAELLLTA CTSDFVIHGT IHGVVHDMEL | |
| QESVITVVAT RVIRQTLPLF QEGSSEGRGQ ASVRTLLRCG VRPGPGSFLF MGWSRFGEAW | |
| LGCAPRFQEF SRVYSAALAA HLNPCEVALD | |
| Codon optimized Meteorin nucleotide sequence | |
| present in constructs pCAn.Meteorin | |
| and pT2.CAn.Meteorin | |
| (SEQ ID NO: 10) | |
| ATGGGCTTTCCCGCTGCCGCCCTGCTGTGCGCTCTGTGCTGCGGACTGCTGGCTCCTGCAGCCAGAGCCG | |
| GCTACAGCGAGGAACGGTGCAGCTGGCGGGGCAGCGGCCTGACCCAGGAACCTGGCAGCGTCGGCCAGCT | |
| CGCACTGGCCTGTGCAGAAGGCGCCGTGGAGTGGCTGTACCCCGCAGGCGCCCTGAGACTGACCCTGGGC | |
| GGACCCGACCCCAGAGCCAGACCCGGCATTGCCTGTCTGAGGCCCGTGCGGCCTTTCGCTGGCGCCCAGG | |
| TGTTCGCCGAGAGAGCCGGCGGAGCCCTGGAACTCCTGCTCGCCGAAGGCCCTGGTCCAGCCGGCGGAAG | |
| ATGCGTGAGATGGGGCCCAAGAGAGCGGAGAGCCCTGTTCCTGCAAGCCACCCCCCACCAGGACATCAGC | |
| AGACGGGTGGCCGCCTTCAGATTCGAGCTGCGGGAGGACGGTAGACCCGAGCTGCCACCTCAGGCCCACG | |
| GACTGGGAGTGGACGGCGCCTGCAGACCCTGTAGCGACGCCGAGCTGCTGCTCGCCGCCTGCACCAGCGA | |
| CTTCGTGATCCACGGCATCATCCACGGCGTGACCCACGACGTGGAGCTGCAGGAAAGCGTCATCACCGTC | |
| GTCGCCGCCAGAGTGCTGAGACAGACCCCCCCTCTGTTCCAGGCCGGCAGAAGCGGCGACCAGGGCCTGA | |
| CCAGCATCCGGACCCCCCTGAGATGCGGCGTGCATCCCGGACCCGGCACCTTCCTGTTCATGGGCTGGTC | |
| CAGATTCGGCGAGGCCCGGCTGGGCTGCGCTCCCCGGTTCCAGGAATTCAGACGGGCCTACGAGGCCGCC | |
| AGGGCCGCTCATCTGCACCCCTGCGAGGTGGCCCTGCATTGA | |
| Consensus sequence, mature Meteorin | |
| (SEQ ID NO: 11) | |
| GYSEXRCSWR GSGLTQEPGS VGQLXLXCXE GAXEWLYPAG ALRLTLGGXD PXXRPXIXCL | 60 |
| RPXRPFAGAQ VFAERXXGXL ELLLAEGXXX AGGRCXRWGP RERRALFLQA TPHXDISRRV | 120 |
| AAFXFELXED XRXEXXPQAX GXGVDGACRP CSDAELLLXA CTSDFVIHGX IHGVXHDXEL | 180 |
| QESVITVVXX RVXRQTXPLF XXGXSXXXGX XSXRTXLRCG VXPGPGXFLF MGWSRFGEAX | 240 |
| LGCAPRFQEF XRXYXAAXXX HLXPCEXALX | 270 |

X is any of the 21 amino acids that can be encoded by DNA.

### REFERENCES

Jørgensen et al., Characterization of Meteorin-An evolutionary conserved neurotrophic factor, J mol Neurosci 2009 Sep; 39 (1-2): 104-116.
Kim Y. S. et al., Coupled Activation of Primary Sensory Neurons Contributes to Chronic Pain. Neuron. 2016, 91(5), 1085-1096.
Nishino et al., “Meteorin: a secreted protein that regulates glial cell differentiation and promotes axonal extension”, EMBO J., 23(9): 1998-2008 (2004).
Johnson et al., Nat. Med., 2:795-799 (1996) Yasuda, Biomed. Ther., 27:1221-1223 (1993)
Hora et al., Bio/Technology, 8:755-758 (1990)
Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462 Reagan-Shaw et al., FASEB J, 22, 659-661 (2007)
Creighton, Proteins-Structure and Molecular Properties, 2nd Ed., W. H. Freeman and Company, New York, 1993.
Wold, F., in Posttranslational Covalent Modification of Proteins, B. C. Johnson, Ed., Academic Press, New York, pp 1-12, 1983
Seifter et al., Meth. Enzymol. 182: 626-646, 1990
Rattan et al., Protein Synthesis: Posttranslational Modifications and Aging, Ann. N.Y. Acad. Sci. 663: 48-62, 1992
Maniatis et al., in Molecular Cloning: A Laboratory Manual, Cold Spring Harbor Laboratory, (NY 1982)
Messing, et al., (Nucleic Acids Res., 9: 309-, 1981),
Maxam, et al., (Methods in Enzymology, 65: 499, 1980).
Banerji et al., Cell 27: 299 (1981)
Corden et al., Science 209: 1406 (1980)
Breathnach and Chambon, Ann. Rev. Biochem. 50: 349 (1981)).
Weiss et al., eds., The molecular biology of tumor viruses: RNA tumor viruses, Cold Spring Harbor Laboratory, (NY 1982)).
Jolly et al., Nucleic Acids Res. 11: 1855 (1983)
Capecchi et al., In: Enhancer and eukaryotic gene expression, Gulzman and Shenk, eds., pp. 101-102, Cold Spring Harbor Laboratories (NY 1991)
Schmidt et al., Nature 314: 285 (1985)
Rossi and deCrombrugghe, Proc. Natl. Acad. Sci. USA 84: 5590-5594 (1987)).
Prockop and Kivirikko, N. Eng. J. Med. 311: 376 (1984)
Smith and Niles, Biochem. 19: 1820 (1980)
de Wet et al., J. Biol. Chem., 258: 14385 (1983)
Gruss et al., Proc. Natl. Acad. Sci. USA 78: 943 (1981)
Benoist and Chambon, Nature 290: 304 (1981)
Fromm and Berg, J. Mol. Appl. Genetics, 1: 457 (1982).
Moreau et al., Nucleic Acids Res. 9: 6047 (1981)
Armelor, Proc. Natl. Acad. Sci. USA 70: 2702 (1973)
Fisher Biotech Source 87-88, Fisher Scientific Co., 1987, pp. 72-75
Sigma Cell Culture Catalog, Sigma Chemical Co., St, Louis, 1991, pp. 162-163
Ventrex Product Catalog, Ventrex Laboratories, 1989.

1. An isolated polypeptide for use in treatment or prevention of chemotherapy-induced neuropathic pain in a subject, said polypeptide comprising an amino acid sequence selected from the group consisting of:
i. the amino acid sequence of SEQ ID NO: 3; and
ii. a biologically active sequence variant of the amino acid sequence of SEQ ID NO: 3, wherein the variant has at least 70% sequence identity to SEQ ID NO: 3.

2. The polypeptide for use according to any one of the preceding claims, wherein said polypeptide is administered simultaneously or intermittently with chemotherapy treatment.

3. The polypeptide for use according to any one of the preceding claims, wherein administration of said polypeptide is initiated prior to initiation of chemotherapy treatment.

4. The polypeptide for use according to claim 3, wherein administration of said polypeptide is initiated at least one day prior to initiation of chemotherapy treatment, such as at least two days prior to initiation of chemotherapy treatment, for example at least three days prior to initiation of chemotherapy treatment, such as at least 4 day, at least 5 day, or at least one week prior to initiation of chemotherapy treatment.

5. The polypeptide for use according to any one of the preceding claims, wherein said polypeptide is administered in conjunction with each administration of chemotherapy.

6. The polypeptide for use according to claim 5, wherein said polypeptide is administered on the same day as initiation of chemotherapy treatment, or at least one day prior to initiation of chemotherapy treatment, such as at least two days prior to initiation of chemotherapy treatment, for example at least three days prior to initiation of chemotherapy treatment, such as at least 4 day, at least 5 day, at least one week prior to initiation of chemotherapy treatment.

7. The polypeptide for use according to any one of the preceding claims, wherein said chemotherapy treatment involves administration of platinum-based drugs, taxanes, epothilones, vinca alkaloids and semi-synthetic analogs, proteasome inhibitors, or immunomodulatory drugs, or combinations thereof.

8. The polypeptide for use according to claim 7, wherein said platinum-based drug is carboplatin, cisplatin, or oxaliplatin.

9. The polypeptide for use according to claim 7, wherein said taxane is paclitaxel or docetaxel.

10. The polypeptide for use according to claim 7, wherein said epothilone is ixabepilone.

11. The polypeptide for the use according to claim 7, wherein said vinca alkaloid is vincristine or vinblastine.

12. The polypeptide for use according to claim 7, wherein the semi-synthetic analog is vinorelbine or eribulin.

13. The polypeptide for use according to claim 7, wherein said proteasome inhibitor is bortezomib.

14. The polypeptide for use according to claim 7, wherein said immunomodulatory drug is thalidomide or lenalidomide.

15. The polypeptide for use according to any one of the preceding claims, wherein the subject to be treated suffers from ovarian cancer, breast cancer, esophageal cancer, pancreatic cancer leukemia, Hodgkin's disease, Wilms' tumor, neuroblastoma, testicular cancer, bladder cancer, or multiple myeloma.

16. The polypeptide for use according to any one of the preceding claims, wherein said polypeptide has at least 70% sequence identity to SEQ ID NO: 3, more preferably at least 75%, more preferably at least 80%, more preferably at least 85%, more preferably 90%, more preferably 95%, more preferably 98% sequence identity to SEQ ID NO: 3.

17. The polypeptide for use according to any one of the preceding claims, wherein the polypeptide comprises the consensus sequence of SEQ ID NO: 11.

18. The polypeptide for use according to any one of the preceding claims, wherein the polypeptide has cysteine residues at positions 7, 28, 59, 95, 148, 151, 161, 219, 243, and 265 relative to the amino acid sequence of SEQ ID NO: 3.

19. The polypeptide for use according to any one of the preceding claims, wherein the polypeptide is a variant polypeptide, wherein any amino acid substitutions are conservative substitutions.

20. The polypeptide for use according to any one of the preceding claims, wherein said polypeptide is capable of forming at least one intramolecular disulphide bridge.

21. The polypeptide for use according to any one of the preceding claims, wherein said treatment results in improvement of allodynia, hyperalgesia, or spontaneous pain.

22. The polypeptide for use according to claim 21, wherein said allodynia is thermal allodynia.

23. The polypeptide for use according to claim 22, wherein said thermal allodynia is cold allodynia or heat allodynia.

24. The polypeptide for use according to claim 21, wherein said allodynia is mechanical allodynia.

25. The polypeptide for use according to claim 21, wherein said hyperalgesia is mechanical hyperalgesia.

26. The polypeptide for use according to any one of the preceding claims, wherein the subject to be treated is mammalian, preferably primate, more preferably human.

27. The polypeptide for use according to any one of the preceding claims, wherein the polypeptide is administered by systemic administration.

28. The polypeptide for use according to any one of the preceding claims, wherein the polypeptide is administered by parenteral injection, preferably subcutaneous injection or intrathecal injection.

29. The polypeptide for use according to any one of the preceding claims, wherein the polypeptide is administered in dosages of 1 μg/kg-10,000 μg/kg, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body.

30. The polypeptide for use according to any one of the preceding claims, wherein said polypeptide is administered at least 1-3 times weekly, such as 2-5 times weekly, such as 3-6 times weekly.

31. The polypeptide for use according to any one of the preceding claims, wherein said polypeptide is administered every other day.

32. The polypeptide for the use according to any one of the preceding claims, wherein said polypeptide is administered daily.

33. The polypeptide for the use according to any one of the preceding claims, wherein administration of said polypeptide is initiated after onset of symptoms of neuropathic pain.

34. The polypeptide for the use according to any one of the preceding claims, wherein administration of said polypeptide is initiated after initiation of chemotherapy treatment.

35. The polypeptide for the use according to any one of the preceding claims, wherein administration of said polypeptide is initiated after initiation of chemotherapy treatment, such as 1 day after, such as 2 days after, such as 3 days after, such as 4 days after, such as 5 days after, such as 8 days after, such as 12 days after initiation of chemotherapy treatment.

36. The polypeptide for the use according to any one of the preceding claims, wherein administration of said polypeptide is initiated after initiation of chemotherapy treatment, such as 1 week after, such as 2 weeks after, such as 3 weeks after initiation of chemotherapy treatment.

37. An isolated nucleic acid molecule for use in treatment or prevention of chemotherapy-induced neuropathic pain in a subject, said nucleic acid molecule comprising a nucleic acid sequence coding for a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3.

38. A vector for use in treatment or prevention of chemotherapy-induced neuropathic pain in a subject, said vector comprising a polynucleotide coding for a polypeptide according to any of the claims 1 to 20.

39. The vector for use of claim 35, further comprising a promoter operably linked to the nucleic acid molecule.

40. The vector for use of any of the preceding claims 38 to 39, wherein the vector is selected from the group consisting of alphavirus, adenovirus, adeno associated virus, baculovirus, HSV, coronavirus, Bovine papilloma virus, and Mo-MLV, preferably adeno associated virus.

41. A method for reducing glutamine synthetase expression in dorsal root ganglion in a subject in need thereof the method comprising administering a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3 thereby reducing expression of glutamine synthetase in dorsal root ganglion.

42. A method for reducing Connexin 43 expression in dorsal root ganglion in a subject in need thereof the method comprising administering a polypeptide comprising an amino acid sequence selected from the group consisting of:
a. the amino acid sequence of SEQ ID NO: 3;
b. a biologically active sequence variant of the amino acid sequence of SEQ ID NO: 3, wherein the variant has at least 70% sequence identity to SEQ ID NO: 3
thereby reducing expression of Connexin 43 in dorsal root ganglion.


SEQUENCE LISTING (11 sequences)

SEQ ID NO: 1 - DNA, 1109 bp, Homo sapiens
  CDS (118)..(999)
gcttcgccggggccgggcggccggcgcccccggctgctcccgccgccgcccggacccgcgccccgccggggcagcggtggtgagagccccgactccccggacgccgcccgccgtgccatggggttcccggccgcggcgctgctctgcgcgctgtgctgcggcctcctggccccggctgcccgcgccggctactccgaggagcgctgcagctggaggggcagcggcctcacccaggagcccggcagcgtggggcagctggccctggcctgtgcggagggcgcggttgagtggctgtacccggctggggcgctgcgcctgaccctgggcggccccgatcccagagcgcggcccggcatcgcctgtctgcggccggtgcggcccttcgcgggcgcccaggtcttcgcggagcgcgcagggggcgccctggagctgctgctggccgagggcccgggcccggcagggggccgctgcgtgcgctggggtccccgcgagcgccgggccctcttcctgcaggccacgccgcaccaggacatcagccgccgcgtggccgccttccgctttgagctgcgcgaggacgggcgccccgagctgcccccgcaggcccacggtctcggcgtagacggtgcctgcaggccctgcagcgacgctgagctgctcctggccgcatgcaccagcgacttcgtaattcacgggatcatccatggggtcacccatgacgtggagctgcaggagtctgtcatcactgtggtggccgcccgtgtcctccgccagacaccgccgctgttccaggcggggcgatccggggaccaggggctgacctccattcgtaccccactgcgctgtggcgtccacccgggcccaggcaccttcctcttcatgggctggagccgctttggggaggcccggctgggctgtgccccacgattccaggagttccgccgtgcctacgaggctgcccgtgctgcccacctccacccctgcgaggtggcgctgcactgaggggctgggtgctggggaggggctggtaggagggagggtgggcccactgctttggaggtgatgggactatcaataagaactctgttcacgcaaaaaaaaaaaaaaaaaaa

SEQ ID NO: 2 - protein, 293 aa, Homo sapiens
MGFPAAALLCALCCGLLAPAARAGYSEERCSWRGSGLTQEPGSVGQLALACAEGAVEWLYPAGALRLTLGGPDPRARPGIACLRPVRPFAGAQVFAERAGGALELLLAEGPGPAGGRCVRWGPRERRALFLQATPHQDISRRVAAFRFELREDGRPELPPQAHGLGVDGACRPCSDAELLLAACTSDFVIHGIIHGVTHDVELQESVITVVAARVLRQTPPLFQAGRSGDQGLTSIRTPLRCGVHPGPGTFLFMGWSRFGEARLGCAPRFQEFRRAYEAARAAHLHPCEVALH

SEQ ID NO: 3 - protein, 270 aa, Homo sapiens
GYSEERCSWRGSGLTQEPGSVGQLALACAEGAVEWLYPAGALRLTLGGPDPRARPGIACLRPVRPFAGAQVFAERAGGALELLLAEGPGPAGGRCVRWGPRERRALFLQATPHQDISRRVAAFRFELREDGRPELPPQAHGLGVDGACRPCSDAELLLAACTSDFVIHGIIHGVTHDVELQESVITVVAARVLRQTPPLFQAGRSGDQGLTSIRTPLRCGVHPGPGTFLFMGWSRFGEARLGCAPRFQEFRRAYEAARAAHLHPCEVALH

SEQ ID NO: 4 - DNA, 1363 bp, Mus musculus
  CDS (84)..(959)
gggcagccgcgccgcgggctgctcgcgctgcggccccgaccctcccggggcagcagtccgaggccccggcgcgtcccctaaccatgctggtagccacgcttctttgcgcgctctgttgcggcctcctggccgcgtccgctcacgctggctactcggaagaccgctgcagctggaggggcagcggtttgacccaggagcctggcagcgtggggcagctgaccctggactgtactgagggcgctatcgagtggctgtacccagctggggcgctgcgcctgaccctgggcggccccgatccgggcacacggcccagcatcgtctgtctgcgcccagagcggcccttcgctggtgcccaggtcttcgctgaacgtatgaccggcaatctagagttgctactggccgagggcccggacctggctgggggccgctgcatgcgctggggtccccgcgagcgccgagcccttttcctgcaggccacaccacaccgcgacatcagccgcagagttgctgccttccgttttgaactgcacgaggaccaacgtgcagaaatgtctccccaggctcaaggtcttggtgtggatggtgcctgcaggccctgcagtgatgccgagctcctcctggctgcatgcaccagtgattttgtgatccacgggaccatccatggggtcgcccatgacacagagctgcaagaatcagtcatcactgtggtggttgctcgtgtcatccgccagacactgccactgttcaaggaagggagctcggagggccaaggccgggcctccattcgtaccttgctgcgctgtggtgtgcgtcctggcccaggctccttcctcttcatgggctggagccgatttggcgaagcttggctgggctgtgctccccgcttccaagagttcagccgtgtctattcagctgctctcacgacccatctcaacccatgtgagatggcactggactgagagacctgggagcaagccctggatggaccttcttctggagatggggtgttggggagggtgatgggagggtgggtgagaagggtgtggctcggatggcatcctggtacccacagtgagctggtagaatactaagtaatctggaccataccagccactgtagtcatggtcttctgtggcaggcagcatacccagctctgtgcctgcctcactttgtctactctccagtctgctgcccttctaacccttcttagcctgctgaccagtgagctcatgttttcctcgaattccagggtgctgctggggttcagagcaaccgtgccgtagtttggaagacttgagctaattgttttttttttgtttgtttttttgtttgtttaaaggtggcctggggggggcggcaaaca

SEQ ID NO: 5 - protein, 291 aa, Mus musculus
MLVATLLCALCCGLLAASAHAGYSEDRCSWRGSGLTQEPGSVGQLTLDCTEGAIEWLYPAGALRLTLGGPDPGTRPSIVCLRPERPFAGAQVFAERMTGNLELLLAEGPDLAGGRCMRWGPRERRALFLQATPHRDISRRVAAFRFELHEDQRAEMSPQAQGLGVDGACRPCSDAELLLAACTSDFVIHGTIHGVAHDTELQESVITVVVARVIRQTLPLFKEGSSEGQGRASIRTLLRCGVRPGPGSFLFMGWSRFGEAWLGCAPRFQEFSRVYSAALTTHLNPCEMALD

SEQ ID NO: 6 - protein, 270 aa, Mus musculus
GYSEDRCSWRGSGLTQEPGSVGQLTLDCTEGAIEWLYPAGALRLTLGGPDPGTRPSIVCLRPERPFAGAQVFAERMTGNLELLLAEGPDLAGGRCMRWGPRERRALFLQATPHRDISRRVAAFRFELHEDQRAEMSPQAQGLGVDGACRPCSDAELLLAACTSDFVIHGTIHGVAHDTELQESVITVVVARVIRQTLPLFKEGSSEGQGRASIRTLLRCGVRPGPGSFLFMGWSRFGEAWLGCAPRFQEFSRVYSAALTTHLNPCEMALD

SEQ ID NO: 7 - DNA, 1026 bp, Rattus norvegicus
  CDS (1)..(876)
atgctggtagcggcgcttctctgcgcgctgtgctgcggcctcttggctgcgtccgctcgagctggctactccgaggaccgctgcagctggaggggcagcggtttgacccaggaacctggcagcgtggggcagctgaccctggattgtactgagggtgctatcgagtggctgtatccagctggggcgctgcgcctgactctaggcggctctgatccgggcacgcggcccagcatcgtctgtctgcgcccaacacggcccttcgctggtgcccaggtcttcgctgaacggatggccggcaacctagagttgctactggccgagggccaaggcctggctgggggccgctgcatgcgctggggtcctcgcgagcgccgagcccttttcctgcaggccacgccacaccgggacatcagccgcagagttgctgccttccaatttgaactgcacgaggaccaacgtgcagaaatgtctccccaggcccaaggttttggtgtggatggtgcctgcaggccctgcagtgatgccgagctccttctgactgcatgcaccagtgactttgtgatccatgggaccatccatggggtcgtccatgacatggagctgcaagaatcagtcatcactgtggtggccactcgtgtcatccgccagacactgccactgttccaggaagggagctcggagggccggggccaggcctccgttcgtaccttgttgcgctgtggtgtgcgtcctggcccaggctccttcctcttcatgggctggagccgatttggcgaagcttggctgggctgcgctccccgcttccaagagttcagccgtgtctattcagctgctctcgcggcccacctcaacccatgtgaggtggcactggactgagagacctgggagcaagccctggatggatcttcctctggggatggggtgttggggaggggtgataggagggtgggtgggaagggtgtggctcagatggcatcctggtacccacagtgaggtggtagaatactaaataacctggatcacacc

SEQ ID NO: 8 - protein, 291 aa, Rattus norvegicus
MLVAALLCALCCGLLAASARAGYSEDRCSWRGSGLTQEPGSVGQLTLDCTEGAIEWLYPAGALRLTLGGSDPGTRPSIVCLRPTRPFAGAQVFAERMAGNLELLLAEGQGLAGGRCMRWGPRERRALFLQATPHRDISRRVAAFQFELHEDQRAEMSPQAQGFGVDGACRPCSDAELLLTACTSDFVIHGTIHGVVHDMELQESVITVVATRVIRQTLPLFQEGSSEGRGQASVRTLLRCGVRPGPGSFLFMGWSRFGEAWLGCAPRFQEFSRVYSAALAAHLNPCEVALD

SEQ ID NO: 9 - protein, 270 aa, Rattus norvegicus
GYSEDRCSWRGSGLTQEPGSVGQLTLDCTEGAIEWLYPAGALRLTLGGSDPGTRPSIVCLRPTRPFAGAQVFAERMAGNLELLLAEGQGLAGGRCMRWGPRERRALFLQATPHRDISRRVAAFQFELHEDQRAEMSPQAQGFGVDGACRPCSDAELLLTACTSDFVIHGTIHGVVHDMELQESVITVVATRVIRQTLPLFQEGSSEGRGQASVRTLLRCGVRPGPGSFLFMGWSRFGEAWLGCAPRFQEFSRVYSAALAAHLNPCEVALD

SEQ ID NO: 10 - DNA, 882 bp, Artificial sequence
  Codon optimised
atgggctttcccgctgccgccctgctgtgcgctctgtgctgcggactgctggctcctgcagccagagccggctacagcgaggaacggtgcagctggcggggcagcggcctgacccaggaacctggcagcgtcggccagctcgcactggcctgtgcagaaggcgccgtggagtggctgtaccccgcaggcgccctgagactgaccctgggcggacccgaccccagagccagacccggcattgcctgtctgaggcccgtgcggcctttcgctggcgcccaggtgttcgccgagagagccggcggagccctggaactcctgctcgccgaaggccctggtccagccggcggaagatgcgtgagatggggcccaagagagcggagagccctgttcctgcaagccaccccccaccaggacatcagcagacgggtggccgccttcagattcgagctgcgggaggacggtagacccgagctgccacctcaggcccacggactgggagtggacggcgcctgcagaccctgtagcgacgccgagctgctgctcgccgcctgcaccagcgacttcgtgatccacggcatcatccacggcgtgacccacgacgtggagctgcaggaaagcgtcatcaccgtcgtcgccgccagagtgctgagacagaccccccctctgttccaggccggcagaagcggcgaccagggcctgaccagcatccggacccccctgagatgcggcgtgcatcccggacccggcaccttcctgttcatgggctggtccagattcggcgaggcccggctgggctgcgctccccggttccaggaattcagacgggcctacgaggccgccagggccgctcatctgcacccctgcgaggtggccctgcattga

SEQ ID NO: 11 - protein, 270 aa, Artificial sequence
  Consensus sequence
  misc_feature (5)..(5): Xaa can be any naturally occurring amino acid
  misc_feature (25)..(25): Xaa can be any naturally occurring amino acid
  misc_feature (27)..(27): Xaa can be any naturally occurring amino acid
  misc_feature (29)..(29): Xaa can be any naturally occurring amino acid
  misc_feature (33)..(33): Xaa can be any naturally occurring amino acid
  misc_feature (49)..(49): Xaa can be any naturally occurring amino acid
  misc_feature (52)..(53): Xaa can be any naturally occurring amino acid
  misc_feature (56)..(56): Xaa can be any naturally occurring amino acid
  misc_feature (58)..(58): Xaa can be any naturally occurring amino acid
  misc_feature (63)..(63): Xaa can be any naturally occurring amino acid
  misc_feature (76)..(77): Xaa can be any naturally occurring amino acid
  misc_feature (79)..(79): Xaa can be any naturally occurring amino acid
  misc_feature (88)..(90): Xaa can be any naturally occurring amino acid
  misc_feature (96)..(96): Xaa can be any naturally occurring amino acid
  misc_feature (114)..(114): Xaa can be any naturally occurring amino acid
  misc_feature (124)..(124): Xaa can be any naturally occurring amino acid
  misc_feature (128)..(128): Xaa can be any naturally occurring amino acid
  misc_feature (131)..(131): Xaa can be any naturally occurring amino acid
  misc_feature (133)..(133): Xaa can be any naturally occurring amino acid
  misc_feature (135)..(136): Xaa can be any naturally occurring amino acid
  misc_feature (140)..(140): Xaa can be any naturally occurring amino acid
  misc_feature (142)..(142): Xaa can be any naturally occurring amino acid
  misc_feature (159)..(159): Xaa can be any naturally occurring amino acid
  misc_feature (170)..(170): Xaa can be any naturally occurring amino acid
  misc_feature (175)..(175): Xaa can be any naturally occurring amino acid
  misc_feature (178)..(178): Xaa can be any naturally occurring amino acid
  misc_feature (189)..(190): Xaa can be any naturally occurring amino acid
  misc_feature (193)..(193): Xaa can be any naturally occurring amino acid
  misc_feature (197)..(197): Xaa can be any naturally occurring amino acid
  misc_feature (201)..(202): Xaa can be any naturally occurring amino acid
  misc_feature (204)..(204): Xaa can be any naturally occurring amino acid
  misc_feature (206)..(208): Xaa can be any naturally occurring amino acid
  misc_feature (210)..(211): Xaa can be any naturally occurring amino acid
  misc_feature (213)..(213): Xaa can be any naturally occurring amino acid
  misc_feature (216)..(216): Xaa can be any naturally occurring amino acid
  misc_feature (222)..(222): Xaa can be any naturally occurring amino acid
  misc_feature (227)..(227): Xaa can be any naturally occurring amino acid
  misc_feature (240)..(240): Xaa can be any naturally occurring amino acid
  misc_feature (251)..(251): Xaa can be any naturally occurring amino acid
  misc_feature (253)..(253): Xaa can be any naturally occurring amino acid
  misc_feature (255)..(255): Xaa can be any naturally occurring amino acid
  misc_feature (258)..(260): Xaa can be any naturally occurring amino acid
  misc_feature (263)..(263): Xaa can be any naturally occurring amino acid
  misc_feature (267)..(267): Xaa can be any naturally occurring amino acid
  misc_feature (270)..(270): Xaa can be any naturally occurring amino acid
GYSEXRCSWRGSGLTQEPGSVGQLXLXCXEGAXEWLYPAGALRLTLGGXDPXXRPXIXCLRPXRPFAGAQVFAERXXGXLELLLAEGXXXAGGRCXRWGPRERRALFLQATPHXDISRRVAAFXFELXEDXRXEXXPQAXGXGVDGACRPCSDAELLLXACTSDFVIHGXIHGVXHDXELQESVITVVXXRVXRQTXPLFXXGXSXXXGXXSXRTXLRCGVXPGPGXFLFMGWSRFGEAXLGCAPRFQEFXRXYXAAXXXHLXPCEXALX